(19) United States
(12) Patent Application Publication
(10) Pub. No.: US 2012/0277619 A1
(43) Pub. Date: Nov. 1, 2012

(54) DETECTING FOOD INTAKE BASED ON IMPEDANCE

(75) Inventors: Warren L. Starkebaum, Plymouth, MN (US); Orhan Soykan, Shoreview, MN (US); Daniel Bloomberg, Minneapolis, MN (US)

(73) Assignee: Medtronic, Inc., Minneapolis, MN (US)

(21) Appl. No.: 13/360,429

(22) Filed: Jan. 27, 2012

Related U.S. Application Data
(60) Provisional application No. 61/480,959, filed on Apr. 29, 2011.

(51) Int. Cl. A61N 1/36; A61B 5/053

(52) U.S. Cl. 600/547; 607/40

(57) ABSTRACT

In some examples, the disclosure relates to a systems, devices, and techniques for monitoring the occurrence of food intake by a patient. In one example, the disclosure relates to a method including determining a phase of tissue impedance at one or more gastrointestinal tract locations of a patient via a medical device, and determining the occurrence of food intake by the patient based on the determined phase of the tissue impedance. In some examples, a medical device may control the delivery of therapy to a patient based on the determination of food intake based on the phase to the tissue impedance.

This application claims the benefit of U.S. Provisional Application Ser. No. 61/480,959 by Starkebaum et al., which was filed on Apr. 29, 2011, and is entitled “DETECTING FOOD INTAKE BASED ON IMPEDANCE.” U.S. Provisional Application Ser. No. 61/480,959 by Starkebaum et al. is incorporated herein by reference in its entirety.

### TECHNICAL FIELD

The disclosure relates to medical devices and, more particularly, medical devices for monitoring food intake of a patient.

### BACKGROUND

Obesity is a serious health problem for many people. Patients who are overweight often have problems with mobility, sleep, high blood pressure, and high cholesterol. Some other serious risks also include diabetes, cardiac arrest, stroke, kidney failure, and mortality. In addition, an obese patient may experience psychological problems associated with health concerns, social anxiety, and generally poor quality of life.

Certain diseases or conditions can contribute to additional weight gain in the form of fat, or adipose tissue. However, healthy people may also become overweight as a net result of excess energy consumption and insufficient energy expenditure. Reversal of obesity is possible but difficult. Once the patient expends more energy than is consumed, the body will begin to use the energy stored in the adipose tissue. This process will slowly remove the excess fat from the patient and lead to better health. Some patients require intervention to help them overcome their obesity. In these severe cases, nutritional supplements, prescription drugs, or intense diet and exercise programs may not be effective.

Surgical intervention is a last resort treatment for some obese patients who are considered morbidly obese. One common surgical technique is the Roux-en-Y gastric bypass surgery. In this technique, the surgeon staples or sutures off a large section of the stomach to leave a small pouch that holds food. Next, the surgeon severs the small intestine a point between the distal and proximal sections, and attaches the distal section of the small intestine to the pouch portion of the stomach. This procedure limits the amount of food the patient can ingest to a few ounces and limits the amount of time that ingested food may be absorbed through the shorter length of the small intestine. While this surgical technique may be very effective, it poses significant risks of unwanted side effects, including malnutrition, and death.

### SUMMARY

The disclosure is directed to medical devices, systems, and techniques to treat one or more patient conditions via a medical device. A medical device may deliver electrical stimulation therapy (e.g., in the form of electrical stimulation pulses or a substantially continuous waveform) via one or more electrodes to one or more tissue sites of a patient to treat one or more patient conditions. In some examples, the medical device may be configured determine the phase of the tissue impedance tissue impedance at one or more locations on the gastrointestinal (GI) tract of the patient. Based on the phase of the tissue impedance, the medical device may detect the occurrence of food intake by the patient. In some examples, the medical device controls the delivery of electrical stimulation (e.g., initiates or suspends stimulation) to the GI tract of the patient based on the detected occurrence of food intake by the patient. Additionally or alternatively, the medical device may store the detected event in a food intake diary, e.g., for later review of the patient's food intake over a period of time by a clinician.

In one aspect, the disclosure is related to a method comprising determining a phase of tissue impedance at one or more gastrointestinal tract locations of a patient via a medical device; and determining the occurrence of food intake by the patient based on the determined phase of the tissue impedance.

In another aspect, the disclosure is related to a medical device system comprising a sensing module configured to sense a signal at one or more gastrointestinal tract locations of a patient; and a processor configured to determine a phase of tissue impedance at the one or more gastrointestinal tract locations, and determine the occurrence of food intake by the patient based on the determined phase of the tissue impedance.

In another aspect, the disclosure is related to a system comprising means for determining a phase of tissue impedance at one or more gastrointestinal tract locations of a patient via a medical device; and means for determining the occurrence of food intake by the patient based on the determined phase of the tissue impedance.

In another example, the disclosure is directed to a non-transitory computer-readable storage medium comprising instructions to cause one or more programmable processors to determine a phase of tissue impedance at one or more gastrointestinal tract locations of a patient, and determine the occurrence of food intake by the patient based on the determined phase of the tissue impedance.

In another example, the disclosure relates to a non-transitory computer-readable storage medium comprising instructions. The instructions cause a programmable processor to perform any part of the techniques described herein.

The details of one or more examples of the disclosure are set forth in the accompanying drawings and the description below. Other features, objects, and advantages of the disclosure will be apparent from the description and drawings, and from the claims.

### BRIEF DESCRIPTION OF DRAWINGS

FIG. 1 is a schematic diagram illustrating an example implantable gastric electrical stimulation system.

FIG. 2 is a block diagram illustrating example components of an implantable gastric electrical stimulator that delivers gastric electrical stimulation therapy.

FIG. 3 is a block diagram illustrating example components of a patient programmer that receives patient input and communicates with a gastric electrical stimulator.

FIG. 4A is a conceptual diagram illustrating example lead including an example electrode positioned on the stomach of the patient.

FIG. 4B is a conceptual diagram illustrating example electrode arrays positioned on the stomach of the patent.

FIGS. 5-7 are flow diagrams illustrating an example technique for detecting food intake of patient.

FIGS. 8-21 are plots and diagrams of various aspects of examples illustrating one or more aspects of the disclosure.

### DETAILED DESCRIPTION

The disclosure is directed to medical devices, systems, and techniques to treat one or more patient conditions via a medical device. A medical device may deliver electrical stimulation therapy (e.g., in the form of electrical stimulation pulses or a substantially continuous waveform) via one or more electrodes to one or more tissue sites of a patient to treat one or more patient conditions. In some examples, the medical device may be configured determine the phase of the tissue impedance tissue impedance at one or more locations on the gastrointestinal (GI) tract of the patient. Based on the phase of the tissue impedance, the medical device may detect the occurrence of food intake by the patient. In some examples, the medical device controls the delivery of electrical stimulation (e.g., initiates or suspends stimulation) to the GI tract of the patient based on the detected occurrence of food intake by the patient. Additionally or alternatively, the medical device may store the detected event in a food intake diary, e.g., for later review of the patient's food intake over a period of time by a clinician.

In general, electrical stimulation therapy may be used to treat a variety of patient conditions related to the GI tract. In some examples, a medical device may generate and deliver gastric electrical stimulation therapy to one or more tissue sites of GI tract to treat a disorder of the GI tract. Gastric electrical stimulation generally refers to electrical stimulation areas of the gastrointestinal tract including the esophagus (including lower and upper esophageal sphincters), stomach (including pylorus), duodenum, small bowel, large bowel, and anal sphincter. Gastric electrical stimulation may be alternatively referred to as gastrointestinal electrical stimulation.

A medical device system for providing gastric electrical stimulation to a patient may include an implantable medical device (IMD) that generates and delivers electrical stimulation pulses or signals to GI tract tissue site(s) via one or more electrodes carried on one or more implantable leads. In some examples, the electrical stimulation may be generated by an external stimulator such as an external trial stimulator. An external stimulator may deliver stimulation to the desired GI tract tissue sites via one or more electrodes carried on one or more percutaneously implantable leads. In other examples, the electrical stimulator may be a leadless electrical stimulator.

Gastric electrical stimulation therapy may be delivered to the gastrointestinal tract, e.g., the stomach and/or small intestine, to treat a disease or disorder such as obesity or gastroparesis. In the case of obesity therapy, for example, electrical stimulation of the stomach may be configured to cause the stomach to undergo a change in gastric muscle tone, which may be indicated by distention of the stomach, and induce a feeling of satiety within the patient. As a result, the patient may reduce caloric intake because the patient has a reduced urge to eat. Alternatively, or additionally, electrical stimulation of the stomach may be configured to induce nausea in the patient and thereby discourage eating. In addition, electrical stimulation of the duodenum may be configured to increase motility in the small intestine, thereby reducing caloric absorption and/or altering the dynamics of nutrient absorption in ways the promote earlier satiation, thereby reducing caloric intake.

In some examples, gastric electrical stimulation therapy may be delivered to the gastrointestinal tract to treat diabetes. For example, the reduction in caloric intake described above may help treat or manage diabetes, such as, e.g., in the case of Type H Diabetes. In addition, gastric stimulation of the stomach and/or duodenum may be configured to delay gastric emptying, slowing the delivery of nutrients into the small intestine following meals, thereby reducing the occurrence of episodes of post-meal hyperglycemia in Type II Diabetic patients or pre-Diabetic patients with impaired glycemic control.

In the case of gastroparesis, gastric stimulation of the stomach and/or duodenum may be configured to increase or regulate motility. Alternatively or additionally, gastric stimulation may result in changes in neural signaling and/or hormonal secretion/signaling that may result in improved glycemia, possibly via changes in insulin secretion and/or sensitivity. In some examples, gastric stimulation of the stomach and/or duodenum may be configured to normalize motility (e.g., by increasing the rate of gastric emptying when a patient has delayed gastric emptying, or retarding the rate of gastric emptying when a patient has rapid gastric emptying). In other cases, gastric stimulation of the stomach may be configured to treat symptoms of gastroparesis (vomiting, nausea, bloating, etc.)

In some cases, it may be desirable to deliver electrical stimulation to the stomach and/or other locations on the GI tract to treat a patient condition in coordination with the intake of food by the patient. Such a process may reduce the amount of energy consumed by a medical device, e.g., as compared a case in which a medical device delivers therapy to a patient on a substantially continuous basis. In some examples, the intake of food may be manually indicated by a patient via a patient programming device. However, using a voluntarily patient controlled device may not always be a solution as patients may either actively choose or forget to manually indicate the intake of food to a medical device. As such, a closed-looped system, in which the onset or offset of feeding could be detected automatically and used to activate a GES device, may be desirable in some cases.

In accordance with one or more examples of the disclosure, a medical device system may be configured to detect the intake of food by a patient based on the phase of tissue impedance sensed at one more locations of the GI tract (such as, e.g., the stomach). The phase of the tissue impedance (which is generally a complex impedance) may refer to the phase shift between the current the voltage. In cases in which the phase of the tissue impedance is measured by application of a current signal, the phase of the tissue impedance may refer to the phase angle between the applied current signal and the corresponding voltage signal.

In some examples, a medical device may be configured to measure the phase of tissue impedance at one or more stomach locations over a period of time to detect phase behavior or indicators that are indicative of food intake by the patient. In some examples, an increase or decrease in the phase of the tissue impedance sensed at a GI tract location within a particular window of time may be an indicator that a patient has ingested food. Additionally or alternatively, particular values or ranges of value of the phase (which may be expressed in terms of phase angle) may be indicative of food intake. When such behavior and/or values are identified in the phase of the tissue impedance, one more processors of a therapy system may determine the onset of food intake by a patient.

In some examples, a medical device may control the delivery of electrical stimulation to the GI tract of the patient based on the detected occurrence of food intake by the patient. For example, when a medical device detects the occurrence of food intake by a patient, the medical device may initiate the delivery of electrical stimulation to the GI tract of the patient or modify one or more parameters of electrical stimulation being delivered to the patient. For example, for an obese or diabetic patient, the medical device may control the delivery of electrical stimulation to induce the feeling of satiety and/or nausea in the patient to discourage the continued intake of food by the patient. By delivering such electrical stimulation to a patient based on the detection of food intake, the medical device may target the timing of the therapy at an instance when the therapy is most effective, e.g., rather than delivering the therapy on a continuous basis or otherwise irrespective of the intake of food by a patient. Additionally, delivering therapy in coordination with food intake rather than on a substantially continuous basis may preserve battery power.

Additionally or alternatively, a medical device may store the detected occurrence of food intake based on the tissue impedance phase in a food intake diary, e.g., for later review of the patient's food intake patterns over a period of time by a clinician. In this manner, for example, a clinician or patient may gauge the effectiveness of therapy designed to reduce the frequency of food intake by the patient.

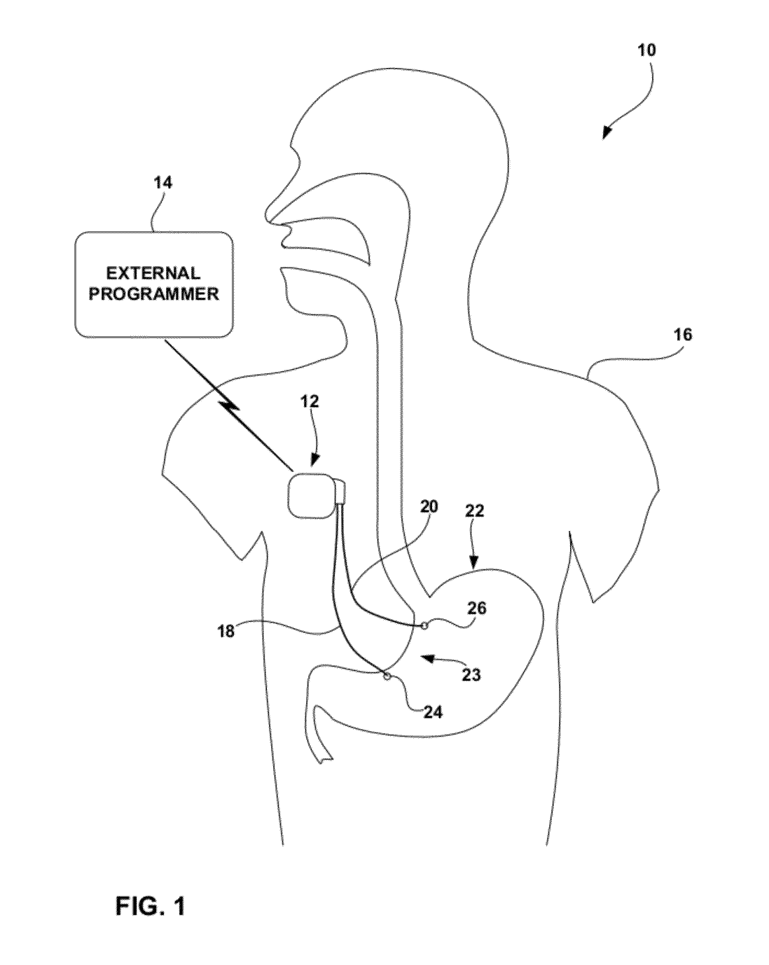

FIG. 1 is a schematic diagram illustrating an example implantable gastric stimulation system 10. System 10 is configured to deliver gastric stimulation therapy to the GI tract of patient 16. Patient 16 may be a human or non-human patient. However, system 10 will generally be described in the context of delivery of gastric stimulation therapy to a human patient, e.g., to treat obesity or gastroparesis, or otherwise control or influence food intake or gastric motility.

As shown in FIG. 1, system 10 may include an IMD 12 and an external patient programmer 14, both shown in conjunction with a patient 16. In some examples, IMD 12 may be referred to generally as an implantable stimulator. Patient programmer 14 and IMD 12 may communicate with one another to exchange information such as commands and status information via wireless telemetry.

IMD 12 may deliver electrical stimulation energy, which may be constant current or constant voltage based pulses, to one or more targeted locations within patient 16 via one or more electrodes 24 and 26 carried on implantable leads 18 and 20. IMD 12 may generate and deliver the electrical stimulation pulses based on the stimulation parameters defined by one or more programs used to control delivery of stimulation energy. The parameter information defined by the stimulation programs may include information identifying which electrodes have been selected for delivery of stimulation according to a stimulation program, the polarities of the selected electrodes, i.e., the electrode configuration for the program, voltage or current amplitude, pulse rate, pulse shape, and pulse width of stimulation delivered by the electrodes. Delivery of stimulation pulses will be described for purposes of illustration. However, stimulation may be delivered by IMD 12 to patient 16 in other forms, such as continuous waveforms. In some examples, system 10 may further include a drug delivery device that delivers drugs or other agents to the patient for obesity or gastric motility therapy, or for other nongastric related therapies. Again, system 10 may use an external, rather than implanted, stimulator, e.g., with percutaneously implanted leads and electrodes.

Leads 18 and 20 each may include one or more electrodes 24 and 26 for delivery of the electrical stimulation pulses to stomach 22. In an example in which leads 18 and 20 each carry multiple electrodes, the multiple electrodes may be referred to as an electrode array. Combinations of two or more electrodes on one or both of leads 18, 20 may form bipolar or multipolar electrode pairs. For example, two electrodes on a single lead may form a bipolar arrangement. Similarly, one electrode on a first lead and another electrode on a second lead may form a bipolar arrangement. Various multipolar arrangements also may be realized. A single electrode 24, 26 on leads 18, 20 may form a unipolar arrangement with an electrode carried on a housing of IMD 12. Although the electrical stimulation, e.g., pulses or continuous waveforms, may be delivered to other areas within the gastrointestinal tract, such as, e.g., the esophagus, duodenum, small intestine, and/or large intestine, delivery of stimulation pulses to stomach 22 will generally be described in this disclosure for purposes of illustration. In the example of FIG. 1, electrodes 24, 26 are placed in lesser curvature 23 of stomach 22. Alternatively, or additionally, electrodes 24, 26 could be placed in the greater curvature of stomach 22 or at some other location of stomach 22.

In some examples, system 10 may be configured to deliver electrical stimulation therapy in a manner that influences that gastric distension of stomach 22 of patient 16. Gastric distention may generally refer to an increase in gastric volume or a relaxation in gastric muscle tone. Hence, a volumetric increase associated with gastric distention may be indicative of a state or relaxation of gastric muscle tone. In general, gastric distention, increase in gastric volume and relaxation of gastric muscle tone may be used interchangeably to generally refer to a relative state of contraction or relaxation of the stomach muscle. In some cases, increased gastric distention may correlate with reduced food intake by a patient.

The state of contraction or relaxation of the stomach muscle may be evaluated using a device called a balloon barostat. The Distender Series II™, manufactured by G&J Electronics, Inc., Toronto, Ontario, Canada, is an example of a balloon barostat system that may be used to diagnose certain gastric motility disorders. Using this system, a balloon is inserted into the stomach, and inflated to a pressure just above the abdominal pressure, referred to the minimum distending pressure. The barostat is configured so that the pressure in the balloon is maintained at a constant pressure. If the state of contraction of stomach muscle decreases, i.e., the state of relaxation of the stomach muscle increases, then the balloon volume will increase. A decrease in the state of stomach muscle contraction, if measured under conditions of constant balloon pressure, indicates a change in gastric muscle tone, i.e., gastric muscle relaxation, and is sometimes referred to as a change in gastric distention, gastric volume, or gastric tone. More particularly, a decrease in muscle contraction corresponds to an increase in muscle relaxation and promotes distention, which may be measure in terms of an increase in gastric volume using balloon barostat evaluation.

Gastric stimulation therapy is described herein in some examples as being provided to cause gastric distention, which may be associated with an increase in gastric volume and an increase in gastric muscle tone relaxation. Alternatively or additionally, gastric stimulation therapy may be delivered by system 10 to induce nausea, cause regurgitation or vomiting (e.g., if too much food is consumed), or cause other actions to treat certain patient disorders. In some examples, gastric stimulation therapy may be delivered by system 10 to prevent regurgitation or reflux (e.g., in the case of gastroesophageal reflux disease (GERD)). In other embodiments, gastric stimulation therapy parameters may be selected to induce or regulate gastric motility (e.g., slow or increase motility), while in other embodiments the gastric stimulation therapy parameters are selected not to induce or regulate gastric motility but to promote gastric distention.

Inducing gastric distention in patient 16 may cause patient 16 to feel prematurely satiated before or during consumption of a meal. Increased gastric distention and volume are generally consistent with a decreased state of stomach muscle contraction, which conversely may be referred to as an increased state of stomach muscle relaxation. While gastric stimulation therapy is shown in this disclosure to be delivered to stomach 22, the gastric stimulation therapy may be delivered to other portions of patient 16, such as the duodenum or other portions of the small intestine.

Gastric distention tends to induce a sensation of fullness and thereby discourages excessive food intake by the patient. The therapeutic efficacy of gastric electrical stimulation in managing obesity depends on a variety of factors including the values selected for one or more electrical stimulation parameters and target stimulation site. Electrical stimulation may have mechanical, neuronal and/or hormonal effects that result in a decreased appetite and increased satiety. In turn, decreased appetite results in reduced food intake and weight loss. Gastric distention, in particular, causes a patient to experience a sensation of satiety, which may be due to expansion of the stomach, biasing of stretch receptors, and signaling fullness to the central nervous system.

In some examples, system 10 may be configured to provide multi-site gastric stimulation to patient 16 to vary the location of electrical stimulation to extend efficacious therapy of stomach 22. Multiple electrodes may be located on stomach 22 and connected to IMD 12. For example, electrodes 24, 26 may be electrode arrays in which IMD 12 may selectively activate one or more electrodes of the arrays during therapy to select different electrode combinations. The electrode combinations may be associated with different positions on the stomach or other gastrointestinal organ. For example, the electrode combinations may be located at the different positions or otherwise positioned to direct stimulation to the positions. In this manner, different electrode combinations may be selected to deliver stimulation to different tissue sites. In some examples, IMD 12 may deliver electrical stimulation to stomach 22 via a single electrode that forms a unipolar arrangement with a reference electrode on the housing of IMD 12.

The selection of electrodes forming an electrode combination used for delivery of electrical therapy at one time may change to a different selection of electrodes forming an electrode combination for delivery of electrical therapy at a different time. The selection may vary between each delivery of stimulation or a predetermined number of delivery periods or total amount of delivery time. The electrical stimulation therapies delivered at respective sites may be configured to produce a substantially identical therapeutic result. The different electrode combinations at each site may provide different stimulation channels. As an example, stimulation delivered via the first and second channels may be configured to produce gastric distention, nausea or discomfort to discourage food intake by the patient. In some cases, the stimulation may be configured to regulate gastric motility. In other cases, the stimulation may be configured to not regulate motility, and instead promote distention, nausea or discomfort.

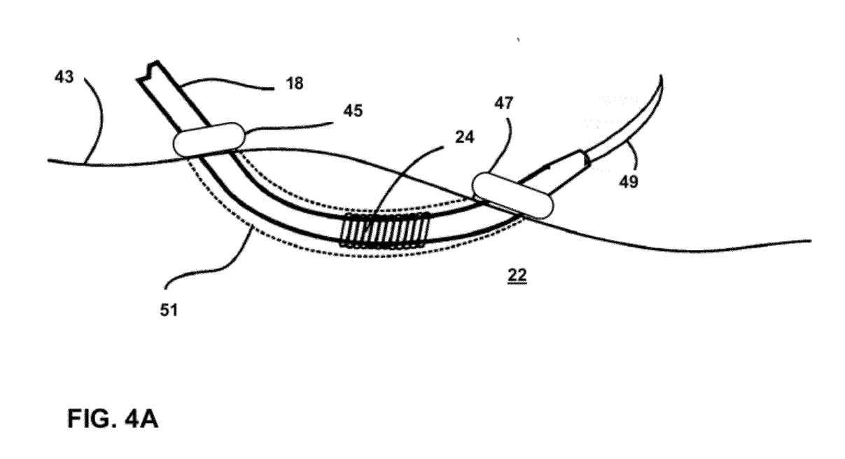

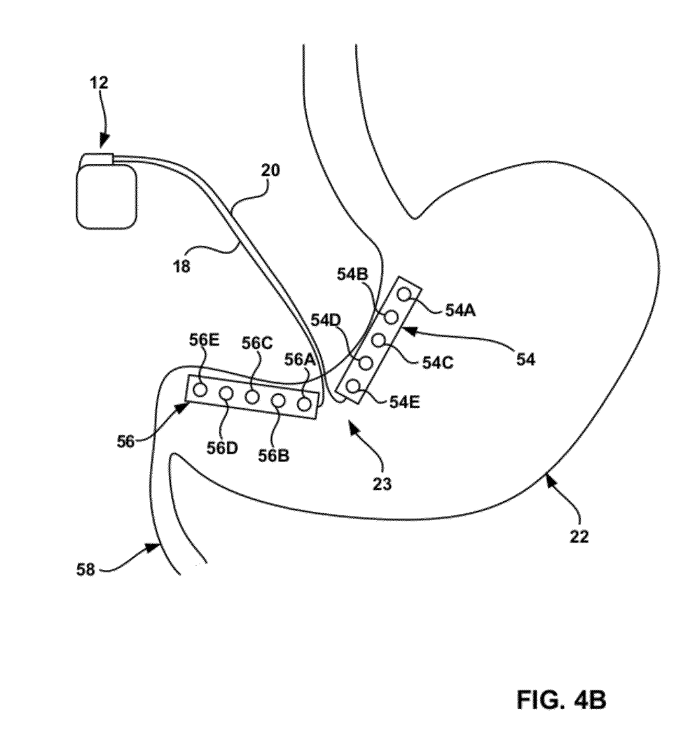

With further reference to FIG. 1, at the outer surface of stomach 22, e.g., along the lesser curvature 23, leads 18, 20 penetrate into tissue such that electrodes 24 and 26 are positioned to deliver stimulation to stomach 22. For example, lead 18 may be tunneled into and out of the wall of stomach 22 and then anchored in a configuration that allows electrode 24 carried on lead 18 to be located within the wall of stomach 22. Electrode 24 may then form a unipolar arrangement with a reference electrode on the housing of IMD 13 to deliver electrical stimulation to the tissue of stomach 22. Such an example is shown in FIG. 4A below.

As described above, the parameters of the stimulation pulses generated by IMD 12 may be selected to cause distention of stomach 22 and thereby induce a sensation of fullness, i.e., satiety. In some embodiments, the parameters of the stimulation pulses also may be selected to induce a sensation of nausea. In each case, the induced sensation of satiety and/or nausea may reduce a patient's desire to consume large portions of food. Alternatively, the parameters may be selected to regulate motility, e.g., for gastroparesis. Again, the stimulation pulses may be delivered elsewhere within the gastrointestinal tract, either as an alternative to stimulation of lesser curvature 23 of stomach 22, or in conjunction with stimulation of the lesser curvature of the stomach. As one example, stimulation pulses could be delivered to the greater curvature of stomach 22 located opposite lesser curvature 23.

In accordance with some examples of the disclosure, IMD 12 may be configured to sense the tissue impedance phase at one or more location along the GI tract, e.g., at one more locations on stomach, esophagus, and/or duodenum. In particular, IMD 12 may monitor the tissue impedance phase at the one or more locations to detect the occurrence of food intake by patient 16. As will be described below, some impedance phase values and/or behavior may be correlated with food intake by patient 16. In some examples, IMD 12 may sense the tissue impedance phase via one or more of electrodes 24, 26 used to deliver electrical stimulation to stomach 22 of patient. Additionally or alternatively, IMD 12 may sense the tissue impedance phase via one or more of electrodes not used to deliver electrical stimulation to stomach 22 of patient. In one example, IMD 12 may be configured to monitor tissue impedance phase at the upper portion (e.g., fundus) of stomach 22 and deliver stimulation therapy to the lower portion (e.g.,antrum) of stomach 22. By identifying the occurrence of food intake by patient 16 based on the sensed tissue impedance phase, IMD 12 may time the delivery of therapy to patient in conjunction with the occurrence of food intake by patient 16. Such a process may be desirable when the delivery of therapy is most effective when delivered in a temporal relationship with the intake of food by patient 16.

IMD 12 may monitor the tissue impedance phase at one more locations along the GI tract where the impedance phase may be indicative of food intake. While examples of the disclosure are primarily described with regard to monitoring tissue impedance phase at one more locations of stomach 22, other GI tract locations for monitoring tissue impedance phased are contemplated, including the esophagus and/or duodenum.

IMD 12 may be constructed with a biocompatible housing, such as titanium, stainless steel, or a polymeric material, and is surgically implanted within patient 16. The implantation site may be a subcutaneous location in the side of the lower abdomen or the side of the lower back. IMD 12 is housed within the biocompatible housing, and includes components suitable for generation of electrical stimulation pulses. IMD 12 may be responsive to patient programmer 14, which generates control signals to adjust stimulation parameters. In some examples, IMD 12 may be formed as an RF-coupled system in which an external controller such as patient programmer 14 or another device provides both control signals and inductively coupled power to an implanted pulse generator.

Electrical leads 18 and 20 are flexible and include one or more internal conductors that are electrically insulated from body tissues and terminated with respective electrodes 24 and 26 at the distal ends of the respective leads. The leads may be surgically or percutaneously tunneled to stimulation sites on stomach 22. The proximal ends of leads 18 and 20 are electrically coupled to the pulse generator of IMD 12 via internal conductors to conduct the stimulation pulses to stomach 22 via electrodes 24, 26.

Leads 18, 20 may be placed into the muscle layer or layers of stomach 22 via an open surgical procedure, or by laparoscopic surgery. Leads also may be placed in the mucosa, submucosa, and/or muscularis by endoscopic techniques or by an open surgical procedure. Electrodes 24, 26 may form a bipolar pair of electrodes. Alternatively, IMD 12 may carry a reference electrode to form an “active can” or unipolar arrangement, in which one or both of electrodes 24, 26 are unipolar electrodes referenced to the electrode on the pulse generator. The housing of IMD 12 may itself serve as a reference electrode for the active can arrangement. A variety of polarities and electrode arrangements may be used. Each lead 18, 20 may carry a single electrode or an electrode array of multiple electrodes, permitting selection of different electrode combinations, including different electrodes in a given electrode array, and selection of different polarities among the leads for delivery of stimulation.

In some examples, IMD 12 may be a leadless implantable device that is attached to the outside of stomach muscle, implanted inside of stomach 22, or inside or outside at any location of the gastrointestinal tract of patient 16. In some examples, such as those in which IMD 12 is implanted inside of stomach 22, IMD 12 may be implanted using an esophageal approach, which may be a relatively simple medical procedure. In either case, IMD 12 may include at least two individual electrodes to deliver the stimulation to stomach 12. In some examples, the housing of IMD 12 may act as one electrode, where at least one non-housing electrode can be an electrically isolated electrode referenced to the housing of IMD 12 to deliver stimulation. In addition to delivering stimulation, one or more of the stimulation electrodes may be used to sense the tissue impedance phase at one or locations of stomach 22, while in other examples, separate electrodes may be dedicated to sensing. IMD 12 may be secured inside or outside at desired position of stomach 22 using any suitable attachment technique, including screwing-in, hooking and clamping of IMD 12.

Patient programmer 14 transmits instructions to IMD 12 via wireless telemetry. Accordingly, IMD 12 includes telemetry interface electronics to communicate with patient programmer 14. Patient programmer 14 may be a small, battery-powered, portable device that accompanies patient 16 throughout a daily routine. Patient programmer 14 may have a simple user interface, such as a button or keypad, and a display or lights. Patient programmer also may include any of a variety of audible, visual, graphical or tactile output media. Patient programmer 14 may be a hand-held device configured to permit activation of stimulation and adjustment of stimulation parameters. In some examples, patient 16 may use patient programmer 14 to manually indicate to IMD 12 the occurrence of food intake. Such an indication by the patient may be used in some examples to verify the identification of food intake by patient 16 based on sensed tissue impedance phase by IMD 12.

Alternatively, patient programmer 14 may form part of a larger device including a more complete set of programming features including complete parameter modifications, firmware upgrades, data recovery, or battery recharging in the event IMD 12 includes a rechargeable battery. Patient programmer 14 may be a patient programmer, a physician programmer, or a patient monitor. In some embodiments, patient programmer 14 may be a general purpose device such as a cellular telephone, a wristwatch, a personal digital assistant (PDA), or a pager.

Electrodes 24, 26 carried at the distal ends of lead 18, 20, respectively, may be attached to the wall of stomach 22 in a variety of ways. For example, the electrode may be formed as a gastric electrode that is surgically sutured onto the outer wall of stomach 22 or fixed by penetration of anchoring devices, such as hooks, needles, barbs or helical structures, within the tissue of stomach 22. Also, surgical adhesives may be used to attach the electrodes. In some cases, the electrodes 24, 26 may be placed in the lesser curvature 23 on the serosal surface of stomach 22, within the muscle wall of the stomach, or within the mucosal or submucosal region of the stomach. Alternatively, or additionally, electrodes 24, 26 may be placed in the greater curvature of stomach 22 and/or fundus such that stimulation is delivered to the greater curvature and/or fundus or tissue impedance phase is sensed at the greater curvature and/or fundus.

In some examples, system 10 may include multiple stimulators 12 or multiple leads 18, 20 to stimulate a variety of regions of stomach 22. Stimulation delivered by the multiple stimulators may be coordinated in a synchronized manner, or performed without communication between stimulators. Also, the electrodes may be located in a variety of sites on the stomach, or elsewhere in the gastrointestinal tract, dependent on the particular therapy or the condition of patient 16. Stimulation delivered by the multiple stimulators may be coordinated in a synchronized manner, or performed independently without communication between stimulators. As an example, one stimulator may control other stimulators by wireless telemetry, all stimulators may be controlled by patient programmer 14, or the stimulators may act autonomously subject to parameter adjustment or downloads from patient programmer 14.

Additionally or alternatively, while examples are described herein with system 10 both delivering stimulation and sensing tissue impedance phase using a single device in the form of IMD 12, in other examples, one more distinct devices, separate from that used to deliver electrical stimulation to patient 16, may be used to sense the tissue impedance phase. In such examples, the sensing device may communicate to the stimulation device when the occurrence of food intake is detected based on the sensed tissue impedance phase, e.g., so that the stimulation device may be control the delivery of stimulation to patient 16 in coordination with the intake of food by patient 16. Such communication may be direct or indirect (e.g., via programmer 14).

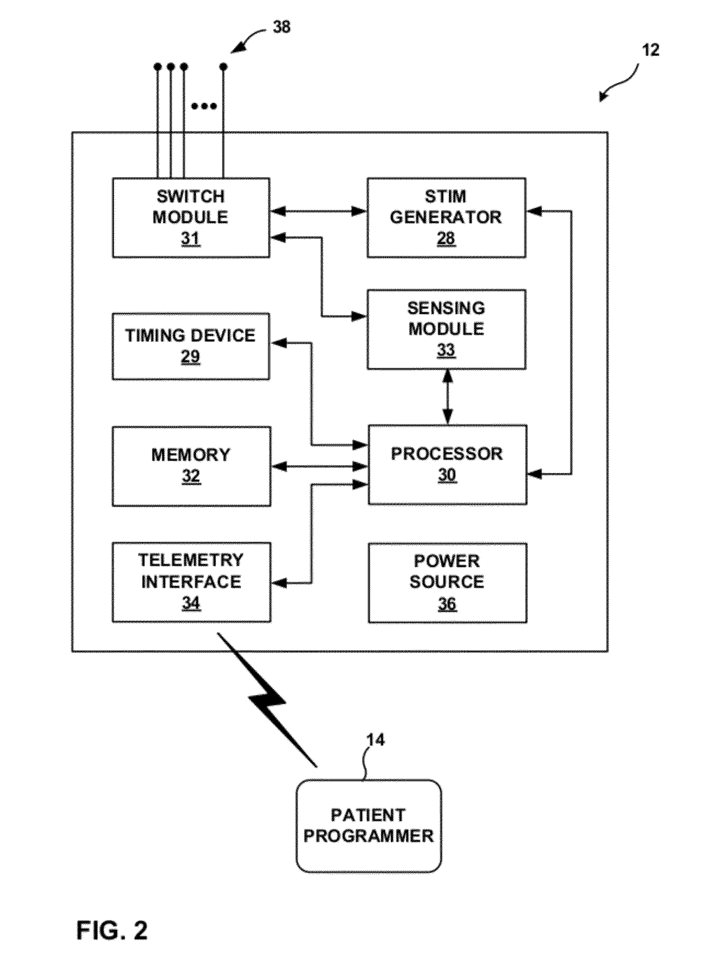

FIG. 2 is a block diagram illustrating example components of IMD 12 that delivers gastric stimulation therapy to patient 16. In the example of FIG. 2, IMD 12 includes stimulation generator 28, sensing module 33, switch module 31, processor 30, memory 32, wireless telemetry interface 34 and power source 36. In some embodiments, IMD 12 may generally conform to the Medtronic Itrel 3 Neurostimulator, manufactured and marketed by Medtronic, Inc., of Minneapolis, Minn. However, the structure, design, and functionality of IMD 12 may be subject to wide variation without departing from the scope of the disclosure. Moreover, in some examples, IMD 12 may not have stimulation capabilities but instead may be used as a monitoring device, e.g., to track the food intake behavior of patient 16 over a period of time.

IMD 12 is coupled to electrodes 38, which may correspond to electrodes 24 and 26 illustrated in FIG. 1, via one or more leads 18, 20. IMD 12 provides stimulation therapy to the gastrointestinal tract of patient 16 and may also sense the tissue impedance phase at one more locations of stomach 22. Processor 30 controls stimulation generator 28 by setting and adjusting stimulation parameters such as pulse amplitude, pulse rate, pulse width and duty cycle, in the case that stimulation generator 28 generates pulses. Alternative embodiments may direct stimulation generator 28 to generate continuous electrical signals, e.g., a sine wave. Processor 30 may be responsive to parameter adjustments or parameter sets received from patient programmer 14 via telemetry interface 34. Hence, patient programmer 14 may program IMD 12 with different sets of operating parameters.

Additionally, processor 30 may control switch module 31 to sense the phase of tissue impedance at one or more GI tract locations with selected combinations of electrodes 38. In particular, switch module 31 may create or cut off electrical connections between sensing module 33 and selected electrodes 38 in order to selectively sense tissue impedance phase at one or more locations of the GI tract of patient. For example, sensing module 33 may include an impedance sensing circuit configured to determine the tissue impedance phase (or phase component of the tissue impedance) via selected electrodes 38. As will be described below, sensing module 33 may be configured to apply one or more signals via one or more of electrodes and then sense a signal generated in response to the applied signal via selected electrodes 38 to determine the phase on the tissue impedance (e.g., based on the time delay between the applied and sensed signals). Sensing module 33 may monitor the phase of tissue impedance on a substantially continuous or periodic basis.

Processor 30 may also control switch module 31 to apply stimulation signals generated by stimulation generator 28 to selected combinations of electrodes 24, 26. In particular, switch module 31 may couple stimulation signals to selected conductors within leads carrying electrodes 38, which, in turn, deliver the stimulation signals across selected electrodes 38. Switch module 31 may be a switch array, switch matrix, multiplexer, or any other type of switching module configured to selectively couple stimulation energy to selected electrodes 22A, 22B and to selectively sense tissue impedance with selected electrodes 24, 26. Hence, stimulation generator 28 is coupled to electrodes 38 via switch module 31 and conductors within one or more leads carrying electrodes 38. In some examples, however, IMD 12 does not include switch module 31. In some examples, IMD 12 may include separate current sources and sinks for each individual electrode (e.g., instead of a single stimulation generator) such that switch module 31 may not be necessary.

Stimulation generator 28 may be a single channel or multi-channel stimulation generator. For example, stimulation generator 28 may be capable of delivering, a single stimulation pulse, multiple stimulation pulses or a continuous signal at a given time via a single electrode combination or multiple stimulation pulses at a given time via multiple electrode combinations. In some examples, however, stimulation generator 28 and switch module 31 may be configured to deliver multiple channels on a time-interleaved basis. For example, switch module 31 may serve to time divide the output of stimulation generator 28 across different electrode combinations at different times to deliver multiple programs or channels of stimulation energy to patient 16.

Processor 30 may control stimulation generator 28 to deliver stimulation to one more location to treat or manage the disorder of patient 12. As described above, in some IMD 12 may be configured to deliver electrical stimulation to the GI tract of patient to treat obesity and/or gastroparesis. In one example, (e.g., to treat gastroparesis), IMD 12 may be configured to deliver electrical stimulation to the greater curvature of stomach 22 (approximately 10 cm proximal to the pylorus). Processor 30 may control stimulation generator 28 to deliver stimulation to the greater curvature with current amplitude of approximately 2 to approximately 15 mA (e.g., approximately 7 mA), a pulse width of approximately 250 to approximately 1,000 microseconds (e.g., approximately 330 microseconds), and a frequency of approximately 10 Hz to approximately 20 Hz (e.g., approximately 14 Hz). In another example, (e.g., to treat obesity), IMD 12 may be configured to deliver electrical stimulation to the lesser curvature of stomach 22 (approximately 2 to 4 cm proximal to the pylorus). Processor 30 may control stimulation generator 28 to deliver stimulation to the greater curvature with current amplitude of approximately 2 to approximately 15 mA (e.g., approximately 7 mA), a pulse width of approximately 1 to approximately 10 milliseconds (e.g., approximately 5 milliseconds), and a frequency of approximately 20 Hz to approximately 40 Hz (e.g., approximately 30 Hz). However, other stimulation sites and/or stimulation parameters values are contemplated.

Memory 32 stores instructions for execution by processor 30, including operational commands and programmable parameter settings. Example storage areas of memory 32 may include instructions associated with one or more therapy programs, which may include each program used by IMD 12 to define parameters and electrode combinations for gastric stimulation therapy. In some examples, memory 32 stores instructions for one or more therapy programs used by processor 30 to control therapy to patient 16 upon detecting the occurrence of food intake by patient 16. Memory 32 may store information defining impedance phase values, behavior, or other parameter used by processor 30 to detect the occurrence of food intake by patient 16 based on sensed tissue impedance phase at one or more locations of stomach 22. For example, such information may include absolute values or ranges of values for tissue impedance phase that are indicative of food intake by patient 16. Such information may also include changes (increase and/or decrease) in tissue impedance phase, e.g. relative to some baseline or threshold value within a given period of time, which may be indicative of food intake by patient 16.

Memory 32 may be considered, in some examples, a non-transitory computer-readable storage medium comprising instructions that cause one or more processors, such as, e.g., processor 30, to implement one or more of the example techniques described in this disclosure. The term “non-transitory” may indicate that the storage medium is not embodied in a carrier wave or a propagated signal. However, the term “non-transitory” should not be interpreted to mean that memory 32 is non-movable. As one example, memory 21 may be removed from IMD 12, and moved to another device. In certain examples, a non-transitory storage medium may store data that can, over time, change (e.g., in RAM).

Processor 30 may access a clock or other timing device 29 within IMD 12 to determine pertinent times, e.g., for enforcement of therapy schedules, lockout periods, and therapy windows, and may synchronize such times with times maintained by patient programmer 14. In some examples, processor 30 may access timing device 29 to determine the time of day or other timing parameter for use by processor 30 to verify a determination of food intake by patient 16 based on sensed tissue impedance phase, as described herein.

Memory 32 may include one or more memory modules constructed, e.g., as random access memory (RAM), read-only memory (ROM), non-volatile random access memory (NVRAM), electrically erasable programmable read-only memory (EEPROM), and/or FLASH memory. Processor 30 may access memory 32 to retrieve instructions for control of stimulation generator 28 and telemetry interface 34, and may store information in memory 32, such as operational information.

Wireless telemetry in IMD 12 may be accomplished by radio frequency (RF) communication or proximal inductive interaction of IMD 12 with patient programmer 14 via telemetry interface 34. Processor 30 controls telemetry interface 34 to exchange information with patient programmer 14. Processor 30 may transmit operational information and receive stimulation parameter adjustments or parameter sets via telemetry interface 34. Also, in some embodiments, IMD 12 may communicate with other implanted devices, such as stimulators or sensors, via telemetry interface 34. In some examples, telemetry interface 34 may be configured to wirelessly communicate with other devices using non-inductive telemetry.

Power source 36 delivers operating power to the components of IMD 12. Power source 36 may include a battery and a power generation circuit to produce the operating power. In some embodiments, the battery may be rechargeable to allow extended operation. Recharging may be accomplished through proximal inductive interaction between an external charger and an inductive charging coil within IMD 12. In other embodiments, an external inductive power supply may transcutaneously power IMD 12 whenever stimulation therapy is to occur.

In the example of FIGS. 1 and 2, IMD 12 includes leads 18, 20. In other embodiments, IMD 12 may be a leadless stimulator, sometimes referred to as a microstimulator, or combination of such stimulators. In this case, the housing of IMD 12 may include multiple electrodes to form electrode combinations for delivery of stimulation to the stomach, intestines, or other organs within patient 16. In additional embodiments, IMD 12 may include one, three, or more than three leads.

Processor 30 may sense the tissue impedance phase at one or more locations of stomach 22 via sensing module 33 and electrodes 38. As described herein, processor 30 and/or other process may receive information defining the tissue impedance phase at one or more locations of stomach 22 and then detect the occurrence of food intake by patient 16 based on the tissue impedance phase. In particular, some values, range of values, and/or behavior of tissue impedance phase may correlate with food intake by patient 16. Processor 30 may also sense tissue impedance magnitude at the one or more locations of stomach 22 via sensing module 33 and electrodes 38. In some examples, processor 30 may use the tissue impedance magnitude to verify a determination of food intake by patient based on sensed tissue impedance phase.

Sensing module 33 may utilize any suitable technique to measure the phase component of tissue impedance between two or more electrodes. In some examples, sensing module 33 take the form of a digital or analog circuit configured to apply a signal across two or more of electrodes 38 and sense a signal in response to the applied signal. For example, sensing module 33 may apply a sinusoidal current across two or more of electrodes 38 and then sense the resulting sinusoidal voltage across the same electrodes to measure the phase shift between the respective signals. Such shift may represent the phase component of the tissue impedance between the electrodes, and in some examples may be expressed in terms of phase angle. In some examples, the phase of the tissue impedance may be represented by the time delay between the applied signal (current or voltage) and the sensed signal (the other of current or voltage). The phase component of impedance may be determined by the sensing module 33 using a suitable measurement circuit based on the principles expressed in Equations 1-6 below. In general, processor 30 may utilize sensing module 33 to determine or otherwise isolate the phase component of the tissue impedance at one or more locations along the GI tract. The determine tissue impedance phase may then be used to determine the occurrence of food intake by patient 12, e.g., using one or more of the techniques described herein. In some examples, sensing module 33 may form a parallel RC circuit configured to determine the phase component of the tissue impedance at one or more GI tract locations.

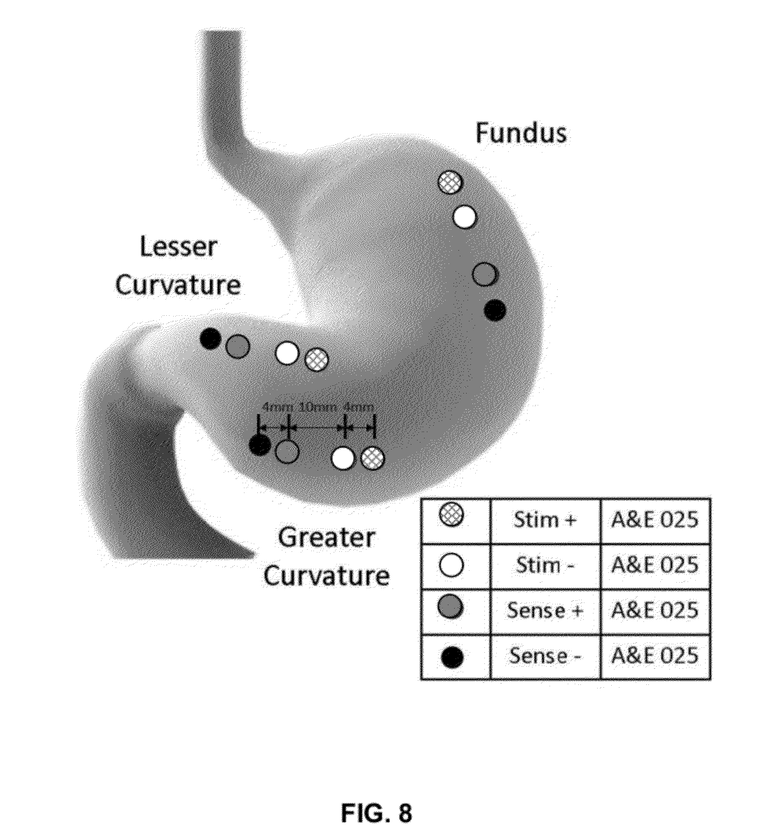

In one example, electrodes 38 may include four electrodes aligned in a substantially linear array at a region of stomach (e.g., fundus, lesser curvature or greater curvature) to sense the tissue impedance phase by way of a quadrapolar impedance measurement. Example electrode configurations for quadrapolar impedance measurements are shown in FIG. 8. Using the two outer electrodes in the array, processor 30 may control stimulation generator 28 to deliver a constant current, sinusoidal wave across the two outer electrodes. Processor 30 may then measure the voltage between the two inner electrodes via sensing module 33. The applied current and the measured voltage may then be used to determine the phase component of the tissue impedance.

For example, in such a case, since the stimulation current is a substantially constant current source, and the voltage is measured, the magnitude and phase angle of the impedance between the electrodes can be calculated by processor based on the following equations:

V=|V|e^{j(ωt+φV)} (1)

I=|I|e^{j(ωt+φI)} (2)

wherein V is the sinusoidal voltage wave and I is the sinusoidal current wave both represented as complex-valued functions in Equations 1 and 2. Impedance, Z, is defined as the ratio of the sinusoidal voltage wave and the sinusoidal current wave of a particular frequency, ω, or

$$Z = \frac{V}{I} . \qquad ( 3 )$$

Substituting the above into Ohm's law:

$$\begin{matrix}\vert V \vert {e}^{j ( ω t + {φ}_{V} )} = \vert I \vert {e}^{j ( ω t + {φ}_{I} )} \vert Z \vert {e}^{j θ} \\ = \vert I \vert \vert Z \vert {e}^{j ( ω t + {φ}_{I} + θ )}\end{matrix} . \qquad ( 4 )$$

As Equation 4 holds true for all t, the magnitudes and phases may be equated to obtain the following:

|V|=|I||Z| (5)

φ_{V}=φ_{I}+ƒ (6)

where Equation 6 defines the phase relationship, where θ is the phase or phase component of the impedance, Φv is the phase of the voltage signal and ΦI is the phase of the current signal.

Again, processor 30 and sensing module 33 may utilize the above relationship between voltage and current to measure phase component of tissue impedance (which may be expressed in terms of phase angle). In some examples, processor 30 and sensing module 33 may utilize a bipolar arrangement described below or any other suitable arrangement capable of determining the phase of tissue impedance at one or more GI tract locations. As will be described below, processor 30 may utilize the measured tissue impedance phase to detect the occurrence of food intake by patient 16.

In some examples, processor 30 of IMD 12 may periodically control stimulation generator 28 to deliver a low level electrical sinusoidal varying measurement current to measure the electrical impedance between the IMD 12 and the electrodes 26 and or 24, or simply between the electrodes 26 and 24. In some examples, the applied current may be on the order of about 50 to 500 micro-amperes and be an alternating current with a frequency of at least 10 Hz, and preferably more than 250 Hz. However, other values for an applied current are contemplate. Additionally, in some examples, a voltage may be applied rather than a current to measure the phase on tissue impedance at one or more locations. When a current is applied, the time delay between the application of the current signal and the sensing of the voltage signal may be measured to estimate the phase of the electrical impedance along the path that the electrical current traverses. Changes in the phase component of electrical impedance may result from the distention of the stomach and may be interpreted by IMD 12 as food intake by the subject. Therefore, the IMD may detect the onset of the meal consumption based on the impedance phase changes and turn on the stimulation (or otherwise control stimulation as described herein). Furthermore, the changes in the phase component of the impedance signal could be used to modulate the duration or the intensity of the stimulation, forming a closed loop control system. In one example, the measurement current is applied between the IMD 12 and electrode 24, while the resulting voltage is measured between the IMD 12 and the electrode 26, forming a three-lead configuration. However, other electrode configurations are contemplated. Moreover, in some examples, IMD 12 may determine the phase of tissue impedance using an applied signal directed towards the measurement of the phase, e.g., as described above. Additionally or alternatively, IMD 12 the signal applied to measure the phase on the tissue impedance may also be delivered to patient 12 for therapeutic purposes.

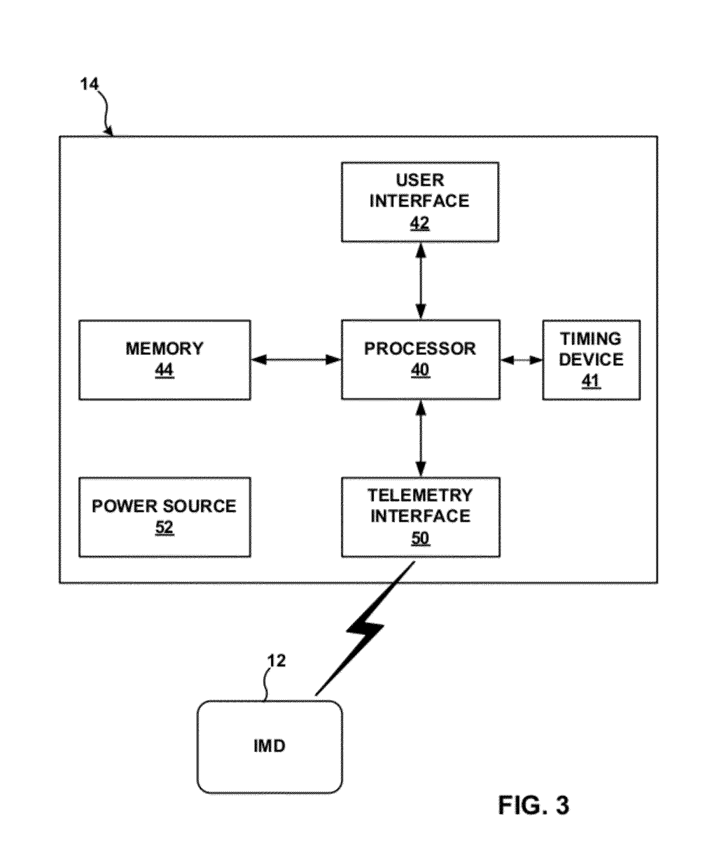

FIG. 3 is a block diagram illustrating example components of patient programmer 14 that receives patient input and communicates with IMD 12. As shown in FIG. 3, patient programmer is an external programmer that patient 16 uses to control the gastric stimulation therapy delivered by IMD 12. Patient programmer 14 includes processor 40, user interface 42, memory 44, telemetry interface 50 and power source 52. In addition, processor 40 may access a clock or other timing device 41 to adhere to lockout periods, therapy windows, and therapy schedules, as applicable. Patient 16 may carry patient programmer 14 throughout therapy so that the patient can initiate, stop and/or adjust stimulation as needed.

While patient programmer 14 may be any type of computing device, the patient programmer may preferably be a hand-held device with a display and input mechanism associated with user interface 42 to allow interaction between patient 16 and patient programmer 14. Patient programmer 14 may be similar to a clinician programmer used by a clinician to program IMD 12. The clinician programmer may differ from patient programmer by having additional features not offered to patient 16 for security, performance, or complexity reasons.

User interface 42 may include display and keypad (not shown), and may also include a touch screen or peripheral pointing devices. User interface 42 may be designed to receive an indication from patient 16 to deliver gastric stimulation therapy. The indication may be in the form of a patient input in the form of pressing a button representing the start of therapy or selecting an icon from a touch screen, for example. In alternative examples, user interface 42 may receive an audio cue from patient 16, e.g., the patient speaks to a microphone in order to perform functions such as beginning stimulation therapy. Patient programmer 14 acts as an intermediary for patient 16 to communicate with IMD 12 for the duration of therapy.

User interface 42 may provide patient 16 with information pertaining, for example, to the status of an indication or a gastric stimulation function. Upon receiving the indication to start stimulation, user interface 42 may present a confirmation message to patient 16 that indicates stimulation has begun. The confirmation message may be a picture, icon, text message, sound, vibration, or other indication that communicates the therapy status to patient 16.

Processor 40 may include one or more processors such as a microprocessor, a controller, a DSP, an ASIC, an FPGA, discrete logic circuitry, or the like. Processor 40 may control information displayed on user interface 42 and perform certain functions when requested by patient 16 via input to the user interface. Processor 40 may retrieve data from and/or store data in memory 44 in order to perform the functions of patient programmer 14 described herein. For example, processor 40 may generate a series of electrical stimulation pulses consistent with one or more examples waveforms described herein based upon instructions stored in memory 44, and processor 40 may then store the selection in memory 44.

Memory 44 may store information relating to the one or more stimulation programs used to define therapy delivered to patient 16. When a new program is requested by IMD 12 or patient 16, parameter information corresponding to one or more of the therapy programs may be retrieved from memory 44 and transmitted to IMD 12 in order adjust the gastric stimulation therapy. Alternatively, patient 16 may generate a new program during therapy and store it within memory 44. Memory 44 may include any volatile, non-volatile, fixed, removable, magnetic, optical, or electrical media, such as a RAM, ROM, CD-ROM, hard disk, removable magnetic disk, memory cards or sticks, NVRAM, EEPROM, flash memory, and the like.

While patient programmer 14 is generally described as a hand-held computing device, the patient programmer may be a notebook computer, a cell phone, or a workstation, for example. In some embodiments, patient programmer 14 may comprise two or more separate devices that perform the functions ascribed to the patient programmer. For example, patient 16 may carry a key fob that is only used to start or stop stimulation therapy. The key fob may then be connected to a larger computing device having a screen via a wired or wireless connection when information between the two needs to be synchronized. Alternatively, patient programmer 14 may simply be small device having one button, e.g., a single “start” button, that only allows patient 16 to start stimulation therapy when the patient feels hungry or is about to eat.

FIG. 4A is a conceptual diagram illustrating lead 18 and electrode 24 positioned to deliver electrical stimulation to stomach 22 of patient 16. Additionally or alternatively, lead 18 and electrode 24 may be positioned to sense the tissue impedance at a location of stomach 22 of patient 16. As shown, a portion of lead 18 is routed into and out the wall of stomach 22. The proximal end of lead 18 includes needle 49, which is used to penetrate the outer surface 43 of stomach 22 and tunnel lead 18 back out of the wall of stomach 22 to form tunnel 51 in the stomach wall. Anchors 45 and 47 fixate lead 18 at the entry and exit points, respectively, to maintain the position of lead 18 within tunnel 51 in the wall of stomach 22.

As shown, lead 18 is positioned within the wall of stomach 22 such that electrode 24 carried on lead 18 is located within tunnel 51 in the wall of stomach 22. Electrode 24 is a coil electrode having a conductive outer surface which is positioned adjacent to tissue of stomach 22. In some examples, to deliver electrical stimulation to stomach 22 from IMD 12, electrode 24 is referenced back to an electrode on the housing of IMD 12 to form a unipolar arrangement. In some examples, lead 18 may carry more than one electrode, each of which may be positioned within tunnel 51 to deliver electrical stimulation using a multipolar (e.g., bipolar) arrangement or unipolar arrangement.

FIG. 4B is a conceptual diagram illustrating example electrode arrays 54 and 56 positioned on stomach 22 of patent 16. As shown in FIG. 4B, electrode arrays 54 and 56 are attached to the outside of stomach 22. Electrode array 54 includes five discrete electrodes 54A, 54B, 54C, 54D and 54E (collectively “electrodes 54”) and electrode array 56 includes five discrete electrodes 56A, 56B, 56C, 56D and 56E (collectively “electrodes 56”). Electrode arrays 54 and 56 are positioned along lesser curvature 23 of stomach 22, but the electrode arrays may be positioned anywhere upon stomach 22 as desired by the clinician. In addition, one or both electrode arrays 54 may be positioned at different sites, such as on the duodenum or elsewhere along the small intestine.

Electrode arrays 54 and 56 are provided in place of electrodes 24 and 26 of FIG. 1. In this manner, electrode arrays 54 and 56 may be used as part of a multi-site electrical stimulation feature to distribute electrical stimulation energy among a larger number of varied tissue sites, instead of concentrating stimulation at a single tissue site. For example, electrode arrays 54, 56 may be used to support selection of different electrode combinations associated with different positions, or tissue sites, on a gastrointestinal organ such as the stomach. Each electrode array 54, 56 may include a plurality of electrodes, e.g., electrodes 54A-54E and electrodes 56A-56E, that may be individually selected to form a variety of electrode combinations that distribute electrical stimulation therapy to different therapy sites. Electrode combinations may include selected electrodes on different leads or the same lead. For example, an electrode combination may combine electrodes from array 54, array 56, or both array 54 and 56, as well as electrodes from other arrays, if provided.

In the example of FIG. 4B, electrode arrays 54 and 56 and electrodes 54A-54E and 56A-56E may not necessarily be sized in proportion to stomach 22. For example, electrode arrays 54 and 56 may be configured to be a smaller size so that the electrodes can be packed into a smaller area of stomach 22. Alternatively, electrode arrays 54 and 56 and their corresponding electrodes may differ in size on stomach 22. For example, electrodes in array 54 may each have a larger surface area than each of the electrodes in array 56. In addition, electrodes 54 may have differing surface areas between each of the electrodes. In this manner, varying electrode surface area may act as an additional anti-desensitization feature to slightly alter the stimulation therapy over time.

IMD 12 may deliver electrical stimulation to stomach 22 using one or more electrodes of electrode arrays 54 and 56. Each of the electrodes in arrays 54, 56 may be coupled to IMD 12 via a respective electrical conductor within leads 18, 20, and may be individually selectable. Each lead 18, 20 may include multiple conductors, each of which is coupled at a distal end to one of the electrodes in a respective electrode array 54, 56 and at the proximal end to a terminal of a switch device by which IMD 12 directs stimulation energy to selected electrodes, e.g., as anodes or cathodes. In some examples, as mentioned above, IMD 12 may deliver stimulation using one electrode from each of electrode arrays 54 and 56, multiple electrodes from one array and a single electrode from another array, or multiple electrodes in a single array.

IMD 12 may cycle through or randomly select different electrodes from each of electrode arrays 54 and 56 to produce different electrode combinations to vary the stimulation tissue sites throughout therapy. In other examples, IMD 12 may deliver stimulation using a combination of any electrodes from only electrode array 54, only electrode array 56, or a combination of electrodes from electrode arrays 54 and 56. In alternative examples, the housing of IMD 12 may also be used as an electrode, e.g., in a unipolar arrangement in conjunction with one or more electrodes carried by one or more leads. The housing of IMD 12 may be referred to as a can electrode, return electrode, or active can electrode, as mentioned above.

While electrode arrays 54 and 56 are shown as each having five electrodes, electrode arrays 54 and 56 may have any number of electrodes desired by the clinician or necessary for efficacious therapy. Electrode arrays 54 and 56 may have differing numbers of electrodes, and IMD 12 may be connected to a different number of electrode arrays, such as only one array or more than three arrays. In addition, electrode arrays 54 and 56 may have corresponding electrodes configured in a different orientation than the linear orientation shown in FIG. 4B. For example, electrode arrays 54 and 56 may have electrodes oriented in a circular pattern, rectangular grid pattern, curved pattern, star pattern, or another pattern that may enhance the anti-desensitization feature of electrode arrays 54 and 56.

In general, multiple electrodes implanted at multiple tissue sites, as shown in FIG. 4B, may permit stimulation to be delivered to different stimulation sites at different times. For example, stimulation having substantially similar parameters or different parameters may be applied to different tissue sites during different therapy windows or therapy schedule time periods such that different tissue sites are stimulated. The stimulation parameters may be selected to achieve similar therapeutic effects, e.g., gastric distention, even though the stimulating is delivered to different tissue sites. Moreover, multiple electrodes implanted at multiple tissue sites, as shown in FIG. 4B, may permit tissue impedance, and the phase of tissue impedance in particular, to be measure at multiple different tissue sites of GI tract at different times.

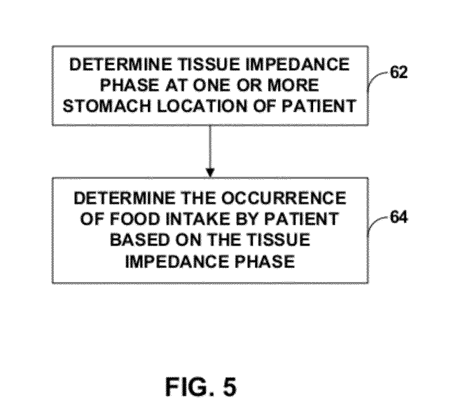

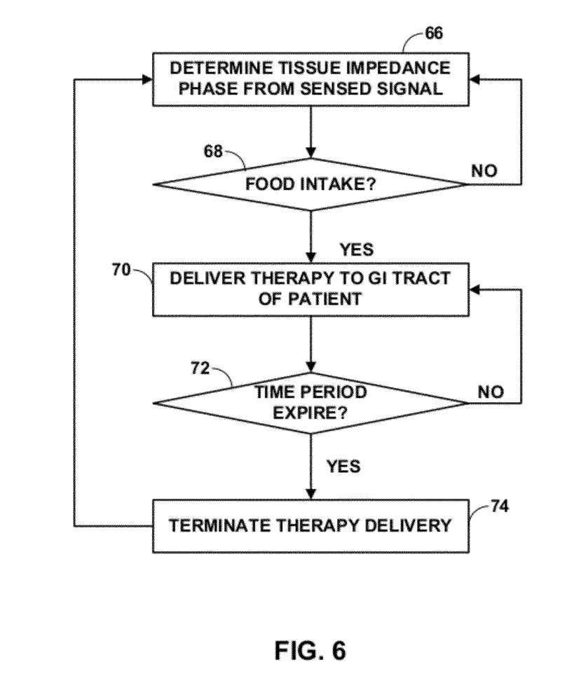

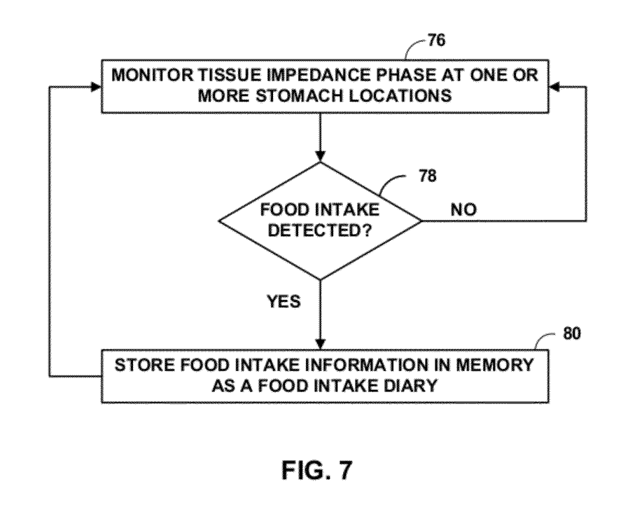

FIG. 5 is a flow diagram illustrating an example technique for detecting food intake of patient 16 based on tissue impedance phase sensed at one or more locations of stomach 22. For ease of illustration, the technique of FIG. 5, as well as FIGS. 6 and 7 are described with regard to therapy system 10 of FIG. 1. However, such techniques may be employed in any suitable system for delivering therapy to patient 16 and/or monitoring food intake of patient 16. Further, processor 30 is described as performing the example techniques of FIGS. 5-7. However, in some examples, all or a portion of such techniques may be performed by one or more other processors, such as, e.g., processor 40 of programmer 14.

As shown in FIG. 5, processor 30 may determine the phase of the sensed tissue impedance, e.g., as described above with regard to FIG. 3 (62). For example, as described above, such information may be sensed via sensing module 33 and one or more of electrodes 38. In some examples, the tissue impedance phase may be calculated by processor 30 in terms of phase angle based on a sensed voltage for a given current delivered by one or more electrodes at a stomach location. The phase component of the tissue impedance may be measured based on the time delay between the applied current and the sensing of the resulting voltage. In some examples, determining the tissue impedance phased may include isolating the phase component from a sensed tissue impedance.

Regardless of the phase of the tissue impedance is determined by processor 30, processor 30 may then determine that occurrence of food intake by patient 16 based on the tissue impedance phase (64). In some examples, processor 30 may make such a determination by comparing the determined tissue impedance phase to one more values or ranges of values for tissue impedance phase stored in memory 32 as being indicative of food intake. For examples, processor 30 may determine that the phase angle of tissue impedance at a particular time is a value or within a range of values that is indicative of food intake by patient 12. In such a case, when processor 30 detects the particular phase angle value via sensing module 33, processor 30 may determine that occurrence of food intake by patient 12.

Additionally or alternatively, the behavior of the sensed tissue impedance phase over a period of time may be stored in memory 32 as being indicative of food intake. For example, a particular increase and/or decrease in the phase component of the tissue impedance within a given period of time, or a particular rate of change above a given threshold, may be determined to be indicative of food intake by patient 12. In some examples, the direction of change, e.g., whether the change is an increase or decrease, of tissue impedance phase may be defined as a indicator of food intake. Regardless of how the methodology for defining indicators of food intake with regard to the phase of tissue impedance, processor 30 may determine whether or not a determined tissue impedance phase indicates the occurrence of food intake at a given time (64).

In some examples, particular indicators of food intake with regard to tissue impedance phase may apply to multiple patients or may be specific to a particular patient disorder. In other examples, the value, range of values, and/or behavior of the tissue impedance phase may be patient specific values. In some examples, indicators of food intake with regard to tissue impedance phase may be determined during a trial period during which the tissue impedance phase is monitored in coordination with one or more known food intake events by patient 12. Based on the behavior of the tissue impedance before, during, and/or after the known food intake events, particular indicators of food intake with regard to tissue impedance phase may be defined and stored in memory 32. During the chronic delivery of therapy and/or chronic monitoring via IMD 12, processor 30 may compare determined tissue impedance phase values or behavior to determine whether or not the determined tissue impedance phase indicate the occurrence of a food intake event. In some examples, indicators of food intake define with regard to tissue impedance phase may be unique to a particular measurement location and/or organ in the GI tract or may apply to multiple locations for measurement in the GI tract. Moreover, as the phase component of tissue impedance may depend on the frequency of a signal, indicators of food intake with regard to tissue impedance phase may be defined for one or frequencies used by sensing module 33 to determine the phase of a tissue impedance.

In some examples, processor 30 may verify a food intake occurrence determined based on the tissue impedance phase with one or more other indicators of food intake. The other indicators may also correlate with food intake and increase a confidence level of the detection of food intake based on tissue impedance phase. For example, processor 30 may determine the magnitude component of the sensed tissue impedance to determine whether or not the magnitude of the tissue impedance is also indicative of the occurrence of food intake by patient 16. The magnitude component of the tissue impedance may be determined by sensing module using any suitable technique known in the art.

Additionally or alternatively, processor 30 may determine the time of day via timing device 29 to determine whether or not the time that processor 30 detected the occurrence of food intake is consistent with the time of day of the detection, e.g., during a time when patient 16 is typically awake versus a time when patient 16 is typically asleep. Additionally or alternatively, processor 30 may determine that time elapsed since the last occurrence of food intake by patient 16 to gauge whether or not it is likely that patient 16 is eating again. In still some examples, processor 30 may verify the occurrence of food intake detected based on tissue impedance phase by determining whether or not patient 16 has manually indicated the occurrence of food intake.

In other examples, processor 30 may determine the occurrence of intake food by patient 12 based only the determined phase of the tissue impedance. For example, processor 30 may make such a determination without looking at another patient parameter indicative of food intake of patient 12. Instead, processor 30 may determine the occurrence of food intake based only on the determined phase of tissue impedance. In such an example, processor 30 may control the delivery of therapy or perform some other action in response to this determination by processor 30.

FIG. 6 is a flow diagram illustrating an example technique for controlling delivery of electrical stimulation based on the detection of food intake by patient 16. As shown in FIG. 6, processor 30 may determine the tissue impedance phase from one or more stomach locations (66), and determine whether or not the phase of the tissue impedance indicates the occurrence of food intake (68). Processor 30 may perform such a determination in a manner substantially the same or similar to that described above with regard to FIG. 5.

If processor 30 determines that the tissue impedance phase does not indicate the occurrence food intake by patient 16, then processor 30 may determine that food intake has not occurred and continue monitoring the tissue impedance phase to detect future food intake by patient 16 (66). Conversely, if processor 30 determines that the tissue impedance phase does indicate the occurrence food intake by patient 16, processor 30 may initiate delivery of stimulation therapy to the GI tract of patient 16 in coordination with the detected food intake (70). As described above, the stimulation delivered to patient 16 may be GES configured to treat one or more patient disorders, such as, e.g., obesity, gastroparesis, or diabetes. In some examples, the electrical stimulation delivered to patient 16 via IMD 12 may be configured to induce satiety or nausea, or regulate gastric motility of the GI tract of patient 16.

For cases in which IMD 12 is actively delivering electrical stimulation to patient 16, e.g., to treat one or more other conditions, processor 30 may adjust the stimulation therapy delivered to patient 16 based on the detection of food intake by patient 16. The adjustment to the stimulation may include an adjustment to one or more stimulation parameters (e.g., amplitude, frequency, pulse width, and/or electrode configuration), and may be used to define a therapy that is appropriate for delivery to patient 12 upon the detection of food intake. In other examples, processor 30 may terminate the delivery of therapy to patient 16 upon detecting the occurrence of food intake, e.g., in cases in which the delivered electrical stimulation is undesirable during the intake of food by patient 16.

Processor 30 may continue to control the stimulation therapy to patient in a manner consistent with the occurrence of food intake of patient for preset period of time (72). In the example of FIG. 6, after the time period has expired, processor 30 may terminate the delivery of therapy to patient 16 (74) and continue monitoring the phase of the tissue impedance for the next occurrence of food intake by patient 16 (66). In cases in which processor 30 adjusted one or more therapy parameters upon the detection of food intake, processor 30 may return to delivering the pre-adjustment therapy to patient 12 after the time period has expired (72). In some examples, processor 30 may resume the delivery of electrical stimulation that was suspended based on the detected intake of food after the expiration of the time period (74).

This time period used in FIG. 6 may be preset by a clinician, e.g., based on the typical time for which the effects of the delivered therapy are experienced by patient 16. In other examples, the time period may be based on indicators that patient 16 is no longer eating. In some examples, processor 30 may continue to monitor the tissue impedance to determine when the modification to therapy made based on the detected onset of food intake should be ended. In some examples, patient 16 may manually indicate to processor 30 via programmer 44 when the modification to therapy made based on the detected onset of food intake should be ended.

Using the technique of FIG. 6, processor 30 may coordinate the delivery of electrical stimulation therapy with the occurrence of food intake by patient 16. In this manner, therapy may be delivered to patient 16 via IMD 12 in closed-loop fashion based on the intake of food by patient 16, as detected by IMD 12 in view of the tissue impedance phase sensed at one or more locations of stomach 22.

FIG. 7 is a flow diagram illustrating an example technique for monitoring the food intake behavior of patient 16 over a period of time. As shown in FIG. 7, processor 30 may monitor the tissue impedance phase at one more locations of stomach 22 (76), and determine whether or not the tissue impedance phase indicates that occurrence of food intake by patient 16 (78). Processor 30 may perform such a determination in a manner substantially the same or similar to that described above with regard to FIG. 5.

If processor 30 determines that the tissue impedance phase does not indicate the occurrence food intake by patient 16, then processor 30 may determine that food intake has not occurred and continue monitoring the tissue impedance phase to detect future food intake by patient 16 (76). Conversely, if processor 30 determines that the tissue impedance phase does indicate the occurrence food intake by patient 16, processor 30 may store food intake information in memory 32 (80) and continue monitoring the tissue impedance phase to detect future food intake by patient 16 (76).

Over a given time period, the food intake information stored by processor 30 may be used to define a food intake diary. Such a food intake diary may be reviewed at a later time by a clinician or other user to review the food intake behavior of patient 16 over a given period of time. Using such information, for example, a clinician or patient may gauge the effectiveness of therapy designed to reduce the frequency of food intake by the patient. The food intake information stored in memory 32 by processor 30 may include information detailing the time of day that the instance of food intake occurred, whether or not patient 16 manually indicated the food intake (e.g., via programmer 44), the length of the food intake event, or other information that may be useful, e.g., to a clinician.

Although the example of FIG. 7 is shown for cases in which IMD 12 monitors food intake of patient 16 but does not delivery therapy to patient 16 in coordination with the detection of food intake, in other examples, such a technique may be combined, for example, with the technique of FIG. 6, for case in which IMD 12 does deliver therapy to patient 16 in coordination with the food intake of patient.

The techniques described in this disclosure may be implemented in hardware, software, firmware or any combination thereof. For example, various aspects of the techniques may be implemented within one or more microprocessors, digital signal processors (DSPs), application specific integrated circuits (ASICs), field programmable logic arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry.

When implemented in software, the functionality ascribed to the systems and devices described in this disclosure may be embodied as instructions on a computer-readable medium such as random access memory (RAM), read-only memory (ROM), non-volatile random access memory (NVRAM), electrically erasable programmable read-only memory (EEPROM), FLASH memory, magnetic media, optical media, or the like. The instructions are executed to support one or more aspects of the functionality described in this disclosure.

### EXAMPLE

An experiment was undertaken to observe changes in the size and shape of the stomach recorded as changes in the impedance between two electrodes placed in the muscle wall of the stomach. Three canines were implanted with impedance sensing electrodes in the fundus, greater curvature, and lesser curvature of stomach. These subjects were given a standard, a high fat, or liquid meal every day while recording the impedance from one set of implanted electrodes over the course of 8 weeks.

To detect eating events and record impedance waveforms, a chronic monitoring procedure was established. The first and fifth weeks of the study, the canines received daily Solid Standard Fat (SSF) meals. The second and sixth weeks of the study, the canines received daily Solid High Fat (SHF) meals. The third and seventh weeks of the study, the canines received daily Liquid Standard Fat (LSF) meals. The fourth and eighth weeks of the study, the canines received daily SSF, SHF, and LSF meals. Impedance measurements were taken in conjunction with the daily meals for weeks 1-3 and weeks 5-7.

As will be described below, analysis of fundic data in canine showed that both the phase and magnitude of the tissue impedance changed its DC setpoint. The signal recorded from the lesser curvature of the stomach also showed a connection between the phase and magnitude of the impedance in which the waveform morphology and correlation between the two components changed with time.

Impedance recordings were made by implanting four unipolar electrodes in a linear array to facilitate a quadraplolar impedance measurement. FIG. 8 is a conceptual diagram illustrating the position of the electrodes for each stomach location. A constant current, approximately 400 microamphere, approximately 12.5 KHz sinusoid was passed from the Stim(+) electrode to the Stim (−) electrode. The voltage in between these two stimulation electrodes was then measured using the Sense(+) and Sense(−) electrodes. Since the stimulation current was a substantially constant current source, and the voltage was being recorded, the instantaneous magnitude and phase angle of the impedance between the electrodes can be calculated as described above with regard to Equations 1-6. All impedance measurements were taken with an Electrobioimpedance Amplifie EBI100C (commercially available from Biopaq Systems, Inc., Goleta, Calif.) impedance amplifier connected to a MP-150 Data Acquisition System (also commercially available from Biopaq Systems, Inc) sampling at a rate of approximately 200Hz. The EBI100C was set to a magnitude range of approximately 1 ohm/volt, with a low pass filter at approximately 100 Hz. Both the magnitude and phase of the impedance signal was captured by a PC running AcqKnowledge 4.0 (also commercially available from Biopaq Systems, Inc).

For a first set of experimental conditions (referred as “Acute Impedance Test”), for each canine, twelve Medtronic Temporary Transvenous Pacing Leads Model 6416 electrodes were implanted via a laparotomy in groups of four in the location and approximate spacing shown in FIG. 8. The leads were placed in groups of four along the fundus, the lesser curvature, and the greater curvature. All the leads were arranged in a linear array in the pattern of positive stimulation lead, positive sensing lead, negative sensing lead, and negative stimulation lead. The interelectrode spacing was approximately 2 mm between the stimulation and sensing leads, and 6 mm-10 mm between the two sensing leads. All twelve leads were implanted before any impedance measurements were made.

Two experiments were performed for each set of leads implanted. First, a baseline recording of impedance was made for the specific set of leads being explored. The stomach was then manually stretched approximately 1 cm with two sets of tweezers. The 1 cm stretch was held for a specific length of time while the impedance recording system continually acquired data. This experiment was designed to simulate the stomach quickly distending after the ingestion of a meal. After the static distention test, the stomach was rapidly distended and compressed approximately 1 cm at a rate of about 1.5 Hz.

An additional bolus ingestion test was also performed in which 50 mL of saline was administered via the esophageal tube. The fundus electrodes were monitored before, during, and after the bolus infusion. This experiment was designed to replicate the fasting meal ingestion sessions that would be implemented in the chronic preparation of the experiment.

For a second set of experimental conditions (referred as “Chronic Impedance Test”), a modified bundle of A&E Medical 025 heart wires were implanted into three canines. The heart wires had their male end pins and needles cut off. A bundle of four wires was then fed through a swelled piece of silicone tubing, and time was allotted for the tubing to constrict around the wires. DB9 male pins were then crimped onto the end of each heart wire. Additional insulation was removed from the distal end of the recording site to allow more contact of the lead to with the tissue.

Leads were implanted in three arrays of four unipolar electrodes, in the locations shown in FIG. 8, via laparotomy. Each bundle of A&E heart wires was implanted in one of the three regions of the stomach. The distal ends of all three bundles were then tunneled out through the abdominal wall, under the skin, and exited just behind the neck of the canine. After the laparotomy was closed, a DB16 connector insert was permanently attached to the pins, and sealed with medical adhesive.

During a recording session, the externalized connector was attached to a TDL build interface cable, which then attached to the TDL fabricated FDA00263 Quick Switch Z Selector Box. As the EBI100C can only record from one set of electrodes at a time, the quick switch box allowed the user to switch the physiological location being recorded from without having to rewire the system.

Each canine was fasted prior to a recording session. The canines were trained to eat their entire daily food intake while in a sling over a one hour period. The three meals (mentioned above) that were tested in the chronic setup were as follows:

1. Solid Standard Fat (SSF)—500 g dry chow (Iams Proactive Health UPC 901401329)

2. Solid High Fat (SHF)—SSF preparation+4 Tbsp. canola oil

3. Liquid Standard Fat (LSF)—16 oz Ensure Shake (Abbot Labs UPC 7007440711)

Baseline recordings were made every day for 10-15 minutes in the sling. After baseline recording, the meal was presented, and the start and stop time of eating was recorded in AcqKnowlege's journal system. Recording sessions lasted one hour total, and were carried out every day.

### Results

### Acute Impedance Test

In the acute impedance test, the impedance recordings from each set of electrodes proved to be stable at around 80±3 Ohms over the course of about 5 minutes of recording in the subject. These recordings were made with the abdominal cavity open and after being exposed to open air for 20 minutes. No contractile activity was apparent in the impedance recordings and no contractions were visualized on the stomach. The lack of contractile activity was most likely due to the deep anesthetic state and the 12 hour previous fast of the canine.

The first experiment conducted on each set of electrodes was the static distension outlined above. The change in gastric impedance could be observed immediately after the stomach was distended. Increasing the distance between the electrodes drove the recorded impedance to a lower magnitude, and had an indeterminate effect on the phase. Rapidly contracting and expanding the stomach rapidly about 1 cm produced a waveform that followed the frequency of the manual manipulations.

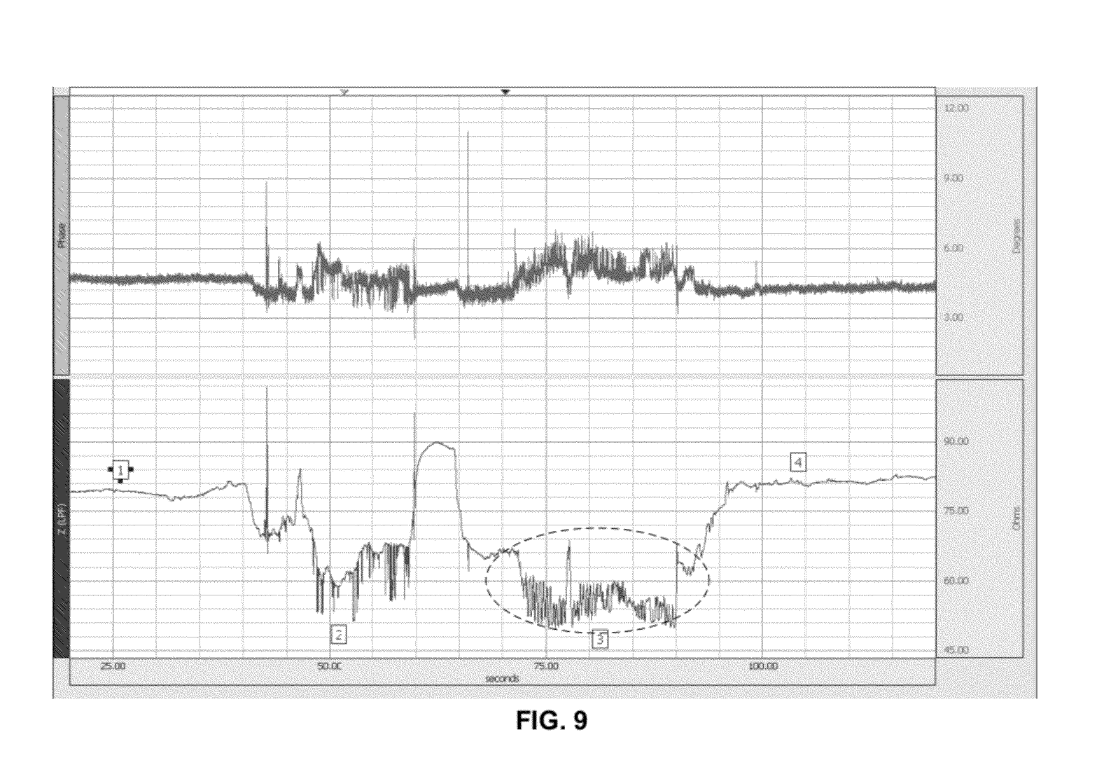

The electrodes in the acute study were implanted through the serosal surface of the stomach, penetrated down to the submucosal layer, traveled approximately 1 cm at that depth, and then exited back up through the serosal surface. The electrodes were not sutured into the gastric wall, and fell out several times in the experiment. FIG. 9 is a plot illustrating the recording from the set of leads implanted in the fundus of the stomach. The phase recording is the top portion and the magnitude recording is the bottom portion. Each numbered square correlates to an event described in Table 1.

TABLE 1

| | | | Phase Average |
|---|---|---|---|
| Marker | Condition | Z Average (Ohms) | (degrees) |
| 1 | Resting | 80 | 4.65 |
| 2 | Manually Distending | 65 | 4.7 |
| | Stomach | | |
| 3 | Rapidly Manually | 50 | 5.2 |
| | Contracting and | | |
| | Relaxing Stomach | | |
| 4 | Resting | 82 | 4.3 |

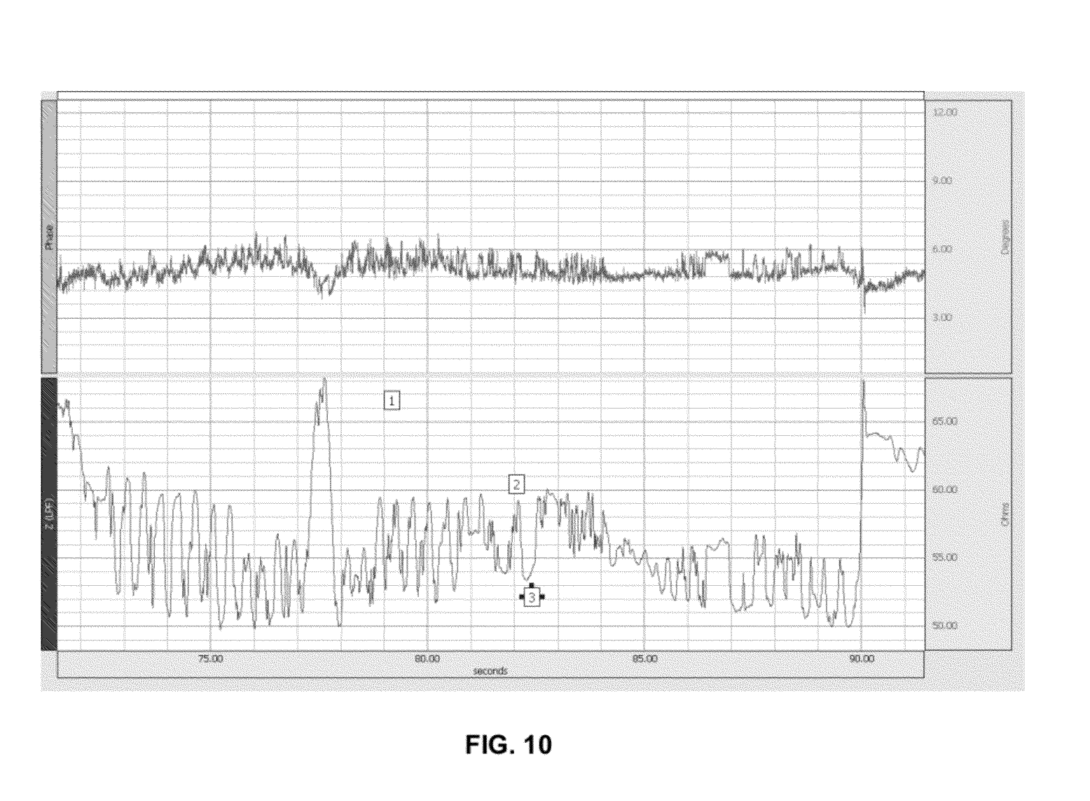

The section circled by the dashed line in the bottom portion of FIG. 9 is enlarged in FIG. 10. Each numbered square in FIG. 10 correlates to an event described in Table 2.

TABLE 2

| | | | Phase Average |
|---|---|---|---|
| Marker | Condition | Z Average (Ohms) | (degrees) |
| 1 | Rapidly Manually | 55.23 (average across | 5.12 |
| | Contracting and | whole section) | |
| | Relaxing Stomach | | |
| 2 | Peak of Contraction | 59.18 | 5.91 |
| 3 | Peak of Distention | 53.21 | 5.23 |

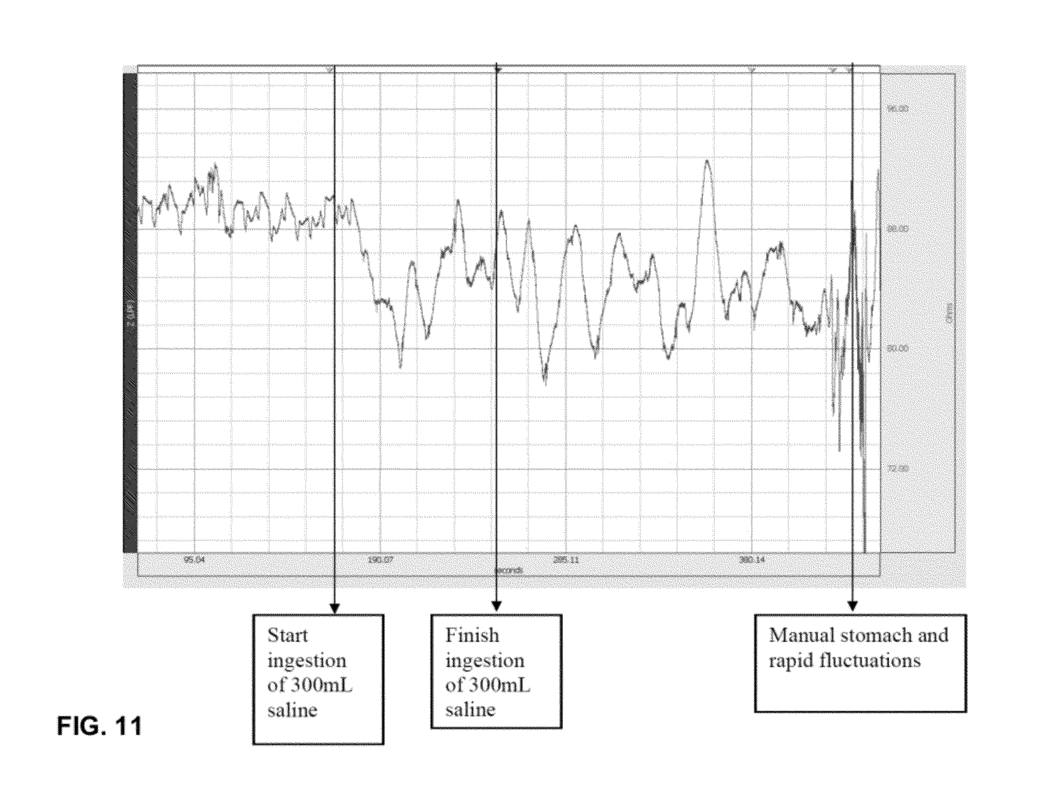

FIG. 11 is a plot of the a second set of recordings made from the fundus approximately 30 minutes after the previous set of recordings shown in FIGS. 9 and 10. A 500 mL bolus of saline was introduced via the canine's stomach tube which caused a decrease in impedance and a distinct change in waveform frequency and amplitude. Table 3 summarizes various impedance parameters before and after the ingestion of the saline bolus.

TABLE 3

| | Before Bolus | After Bolus | Unit |
|---|---|---|---|
| Mean | 89.49338 | 84.67771 | Ohm |
| Average P-P | 5.91362 | 15.12734 | Ohm |
| Average Frequency | 0.01175 | 0.00548 | Hz |
| Min | 86.84708 | 77.67805 | Ohm |
| Max | 92.7607 | 92.8054 | Ohm |

### Chronic Impedance Test

The chronic recording results varied greatly from animal to animal, but remained relatively constant day to day within the same recording site of each animal. A summary of the data collected over the course of the study can be found in Table 4 below. Canine 1 suffered a lead dislodgment nine days into the recording sessions. Several repair attempts were made but the data collected after the lead dislodgment proved unreliable and unstable from day to day. That data collected after the lead dislodgement is not included in the table below.

TABLE 4

| | | | | | | Lesser | | | Greater | | |
|---|---|---|---|---|---|---|---|---|---|---|---|
| | Name | Animal | Fundus Trials | | | Curvature Trials | | | Curvature Trials | | |
| Abbreviation | Abbreviation | ID # | SSF | SHF | LSF | SSF | SHF | LSF | SSF | SHF | LSF |
| Canine 1 | FOL | 338081 | 5 | 0 | 0 | 2 | 1 | 0 | 1 | 0 | 0 |
| Canine 2 | DOV | 338070 | 5 | 3 | 4 | 4 | 5 | 4 | 2 | 2 | 2 |
| Canine 3 | AVA | 338113 | 4 | 4 | 4 | 4 | 4 | 4 | 2 | 2 | 2 |
| | Total | | 14 | 7 | 8 | 10 | 10 | 8 | 5 | 4 | 4 |

Fundus—The set of electrodes implanted in the fundus of canine 1 showed the quickest and most clear indicator of an eating event. In canine 2, across all days of fundus SSF recordings, the stomach impedance (SI) increased by 11±1.3 Ohms, coupled with a negative phase shift of 2.1±0.23°. The delta in both the impedance and phase after an eating event was amplified in the SHF meal, and amplified further in the LSF meal. The magnitude of the impedance change in the LSF meal was 52±5 ohms, while the phase shift became 2.6±0.23 degrees.

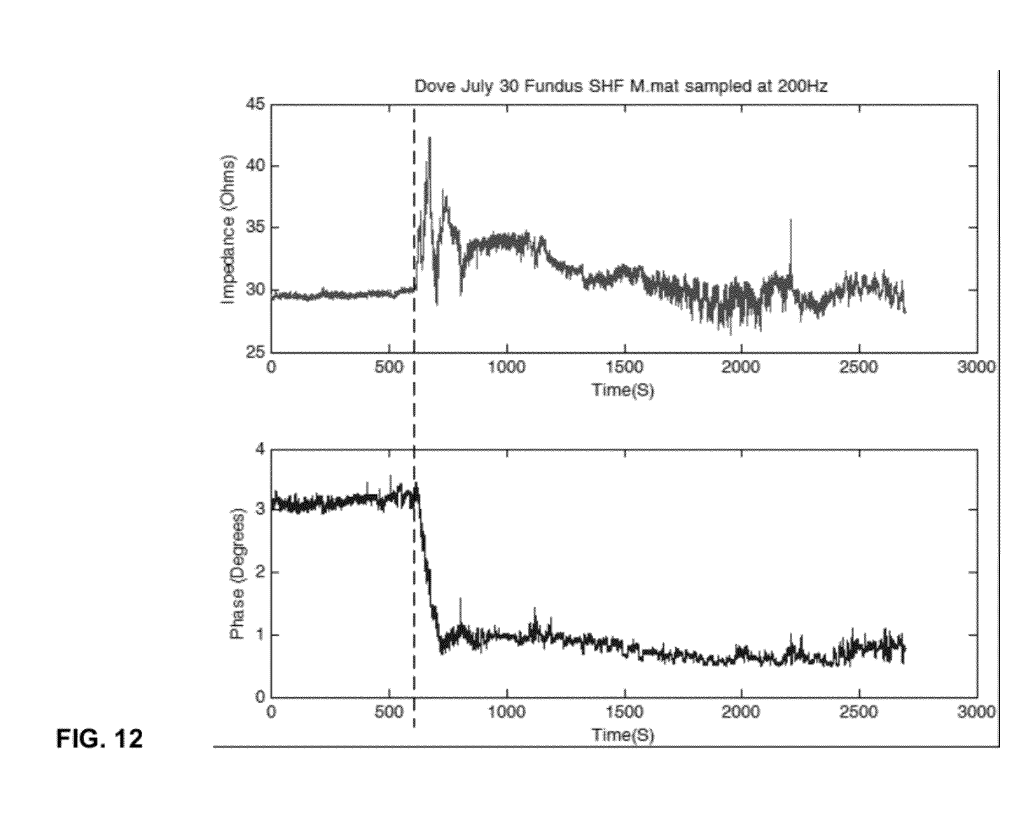

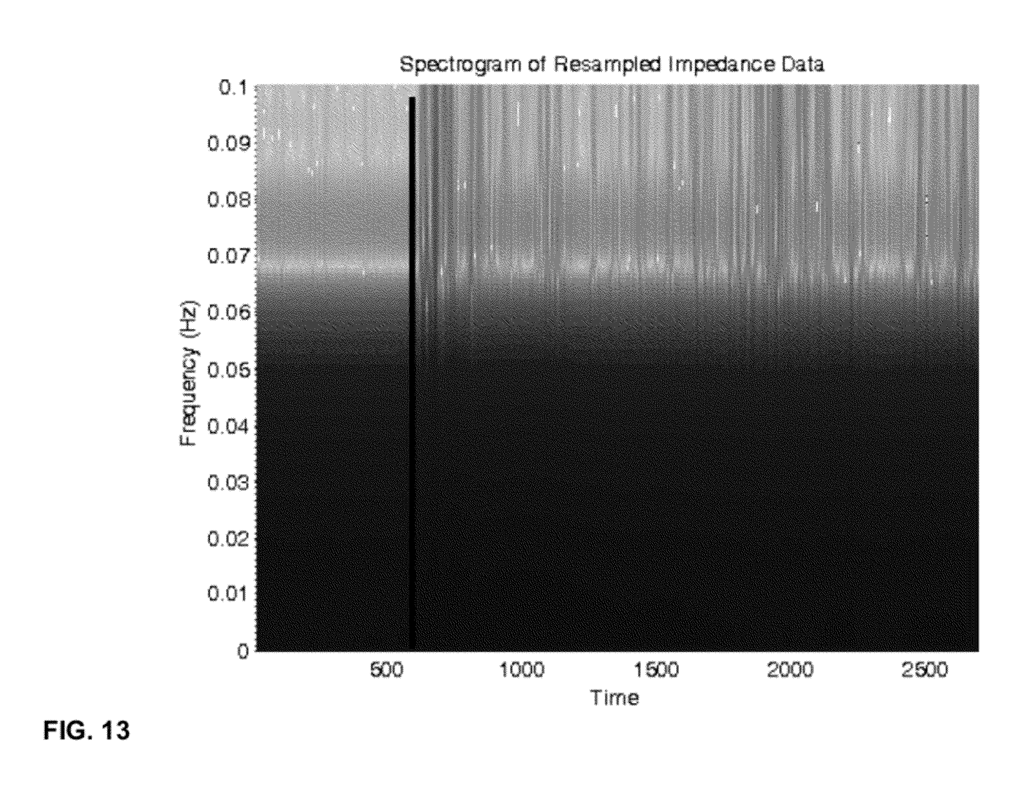

FIG. 12 is a plot of example magnitude and phase of the impedance recorded from the fundus of canine 2. The dashed, vertical line represent the time the meal was delivered. FIG. 13 is a spectrogram of the recorded data showing a quick rise in the impedance magnitude of manifests quickly as an amplification in high frequency components. All the fundic recordings from canine 2 followed the pattern shown in FIGS. 12 and 13. The rapid upstroke in phase if the tissue impedance can be seen as in the frequency spectrum as an amplification of the higher frequency components. The change in both frequency and phase began to occur within about 1 minute of the eating event. The tissue impedance phase decreased in conjunction with the intake of food in FIG. 12 from approximately 3 degrees to approximately 1 degree over a period of approximately 100 seconds. Hence, such phased behavior may be indicative of food intake. Such an indicator may be used by IMD 12 or other device to detect the occurrence of food intake as described above.

The patterns observed in the fundus of the canine 2 were not replicated in the other two subjects. Canine 1 exhibited evidence of the phase and magnitude components of tissue impedance changing by a slow varying DC offset, but that pattern was only observed in two data sets before the lead dislodgment occurred. Canine 3's fundic recordings more closely resembled data recorded from the greater curvature. A detailed explanation of that pattern can be found in the greater curvature discussion below.

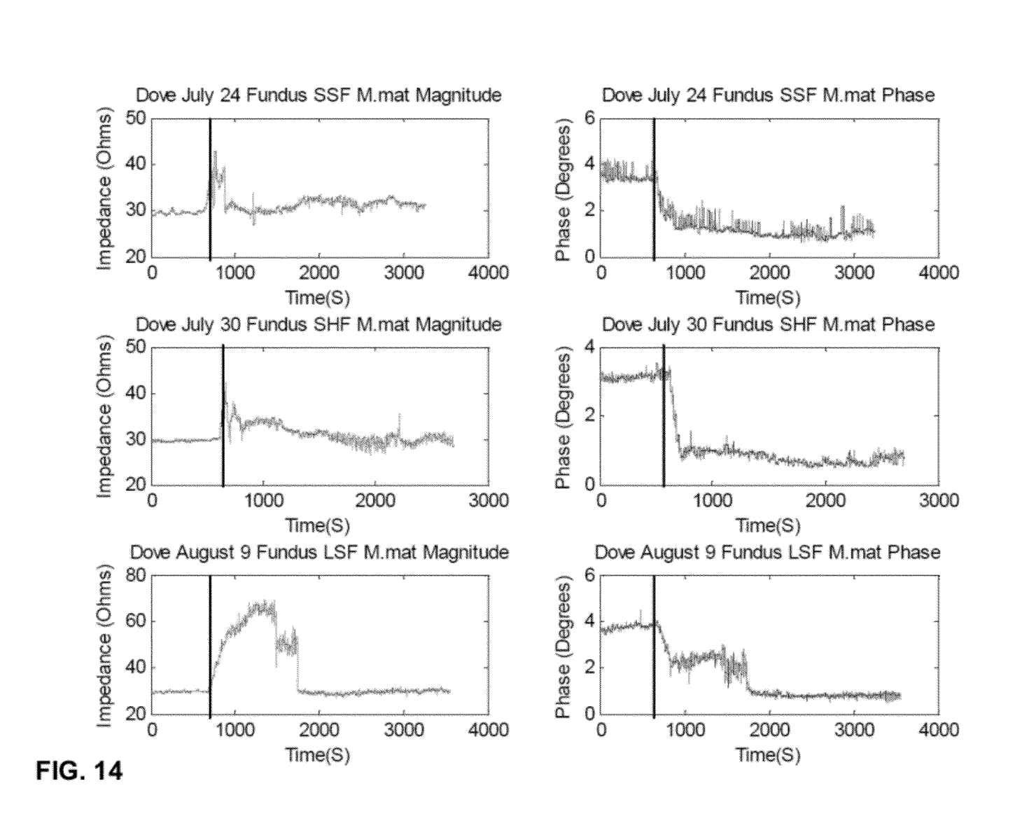

FIG. 14 shows various plots of the magnitude and phase of the impedance recorded from the fundus for each type test meal. The meal modulates the amplitude and duration of the resulting response. The liquid meal had the most significant impact on the change in magnitude of the SI. The onset of feeding in each plot of FIG. 14 is indicated by the black vertical line. Again, similar to that of FIG. 12, the tissue impedance phase decreased in conjunction with the intake of food in each case shown in FIG. 14. Hence, such phased behavior may be indicative of food intake. Such an indicator may be used by IMD 12 or other device to detect the occurrence of food intake as described above.

Lesser Curvature—The lesser curvature, in some examples, may be a location effective for the delivery of GES therapy. As such, electrodes for GES stimulation may be able to serve as sensing electrodes. The data recorded from the lesser curvature was the most consistent of any physiological recording site across all three canines. The response to an eating event in the lesser curvature manifested itself more as a change in impedance waveform morphology, with a small effect on amplitude of the wave. The phase component of the impedance also responded to an eating event by becoming correlated or anti-correlated with the magnitude waveform. The effect of changing the type of meal is less clear in the lower curvature. Since these waveforms more closely correspond to the digestive contractility of the antrum, altering the meal may alter the length of the digestive event, and therefore the length of the modulated waveform activity.

FIG. 14 shows various plots of the magnitude and phase of the impedance recorded from the lesser curvature made on different days, with different meal types, in two distinct canines. The data of FIG. 14 shows that the main features, such as a change in waveform morphology and the net delta of the SI signal, does not change much from subject to subject. Feeding began at approximately 1000 seconds in all trials.

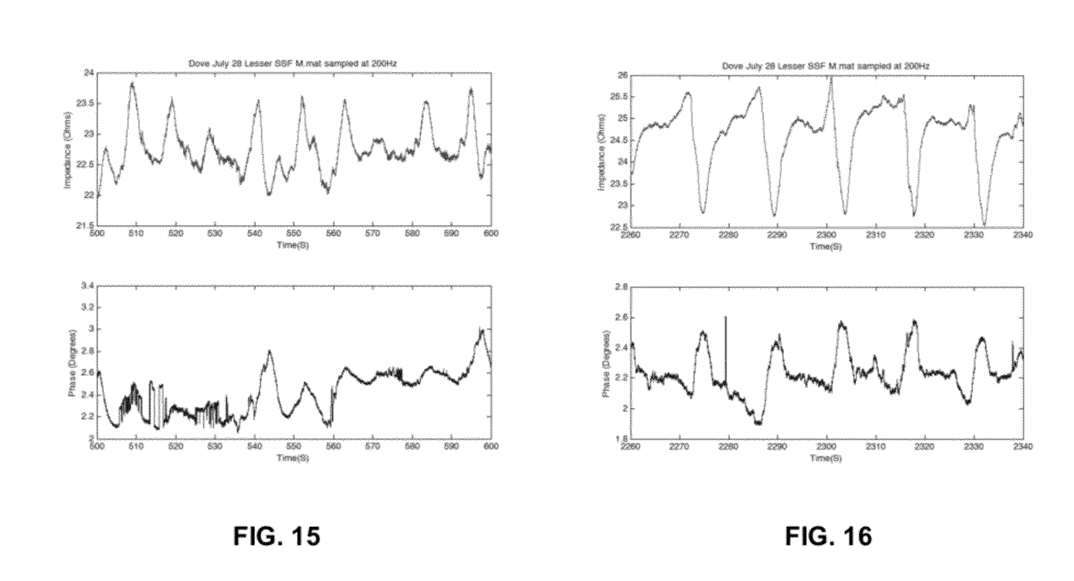

The change in the morphology of the SI waveform can be seen in FIGS. 15 and 16. FIG. 15 shows pre-prandial recordings magnitude and phase from canine 2 over the course of 100 seconds. FIG. 16 shows pre-prandial recordings of magnitude and phase of tissue impedance from the same canine in the same trial. An increase in wave amplitude, change in morphology, and correlation of the phase to magnitude is shown. The peaks in the magnitude signal become much broader after a meal, and the total amplitude change increases. The frequency of the contractions appears to be relativity unchanged from the pre meal state. As shown, the phase component of the tissue impedance also goes from being random spiking activity to a correlated sinusoidal like pattern. This pattern is aligned anti-correlated with the phase magnitude signal.

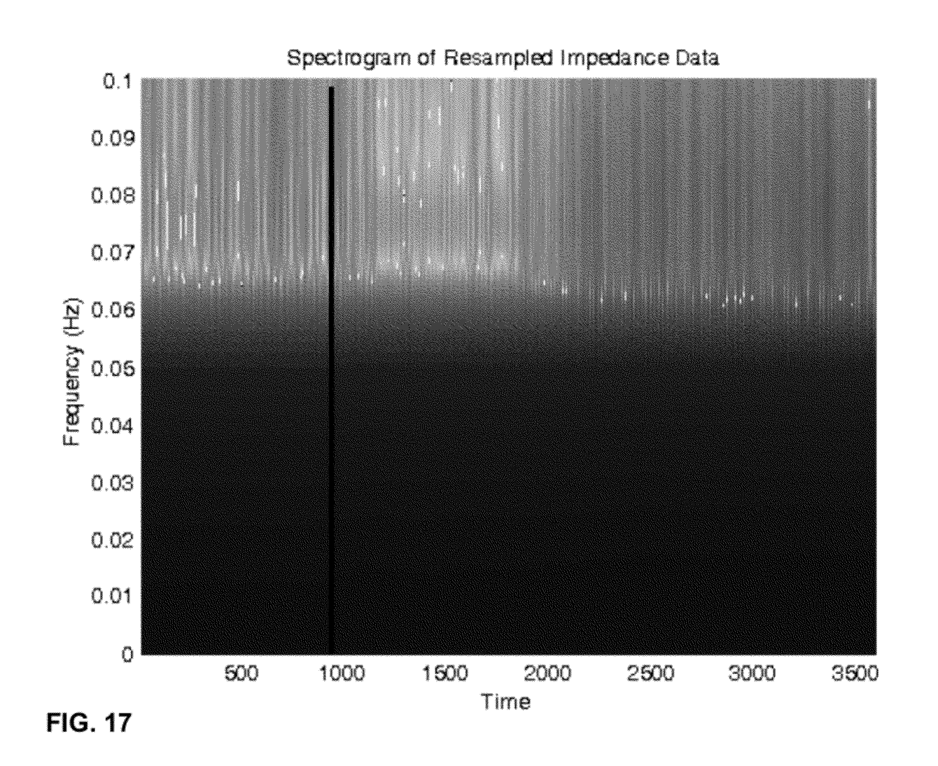

FIG. 17 is a spectrogram of the recorded data from the lesser curvature. As shown, it took approximately 6 minutes to see the effect of the waveform change in the frequency spectrum. The spectrogram clearly shows a change in the higher frequency component about 4 to 6 minutes after the onset of feeding. This behavior was observed in both canine 2 and 3 and reproducible across all lesser curvature data sets.

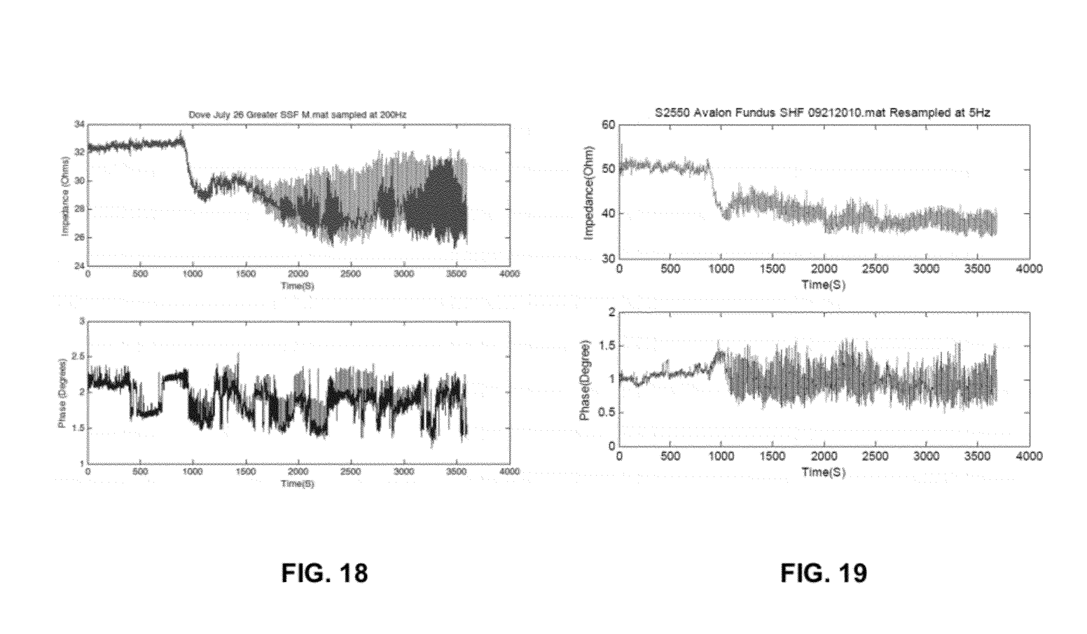

Greater Curvature—The greater curvature is another potential for GES stimulation. The greater curvature signals behaved in a similar manner as signals from the lesser curvature. The changes in the waveform are marked by a change in waveform morphology, amplitude, and frequency. The phase signal also becomes anti-correlated to the magnitude after the onset of the meal, and may be an indicator of food intake Stomach impedance waveforms in the greater curvature had similar characteristics across all three canine subjects. The recordings captured from the fundus of canine 3 resemble the greater curvature recordings from the other two canines. The phenomenon is consistent across all meal types and recording sessions. 101361 An example of a waveform captured from the fundus of canine 3 is shown juxtaposed to a greater curvature recording from canine 2 in FIGS. 18 and 19. FIG. 18 shows a typical recording for the magnitude and phase from the greater curvature of canine 2. FIG. 19 shows a typical recording for the magnitude and phase components of tissue impedance from the fundus of canine 3. The magnitude and phase traces follow a similar pattern in each case, and exhibit the same morphology changing characteristics. Feeding in the examples began at approximately 900 seconds.

### Discussion

Based on the amount of data collected in the experiments, feeding detection based on changes in the phase of tissue impedance may be possible. In some examples, feeding detection based on the phase of tissue impedance from electrodes in the fundus appears may be more easily detectable than distally recorded signals. The change tends to be a clear static shift significantly above the previous time windows standard deviation.

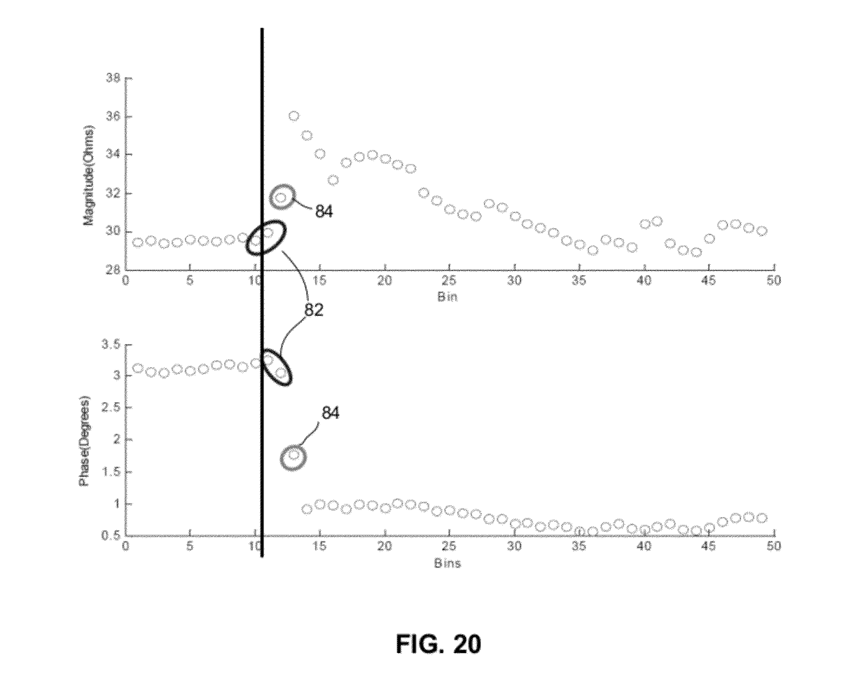

FIG. 20 is a plot of the moving average of data recorded in the fundus of canine 2 with a SSF meal. The data is binned using 1 minute rectangular window. Circles 82 and circle 84 indicate deviations in the mean of the data above the standard deviation of the previous time bin. This strategy was used to identify the onset of feeding in this dataset.

As shown in FIG. 20, both the magnitude and phase of the impedance signal change with contractions of the stomach. The change in phase was unexpected and may represent a decrease in the capacitive effects of the stomach tissue. This decrease may be correlated with the motion of the stomach wall, but it is not clear how the changing geometry of the gastric wall impacts the impedance signal. In the Acute Impedance Test, decrease in impedance magnitude was observed when the stomach was distended. However, in the chronic preparation, both increases and decreases in the magnitude of impedance were observed from the various recording locations.

Changes in real and imaginary components of impedance were observed from electrodes placed along the lesser and greater curvature as well. These changes where observed on a small time scale of 10 seconds, and were reflected in as changes in the morphology and amplitude of the time varying impedance signal. A result of the onset of feeding was the correlation of the phase component to the magnitude component of the impedance signal.

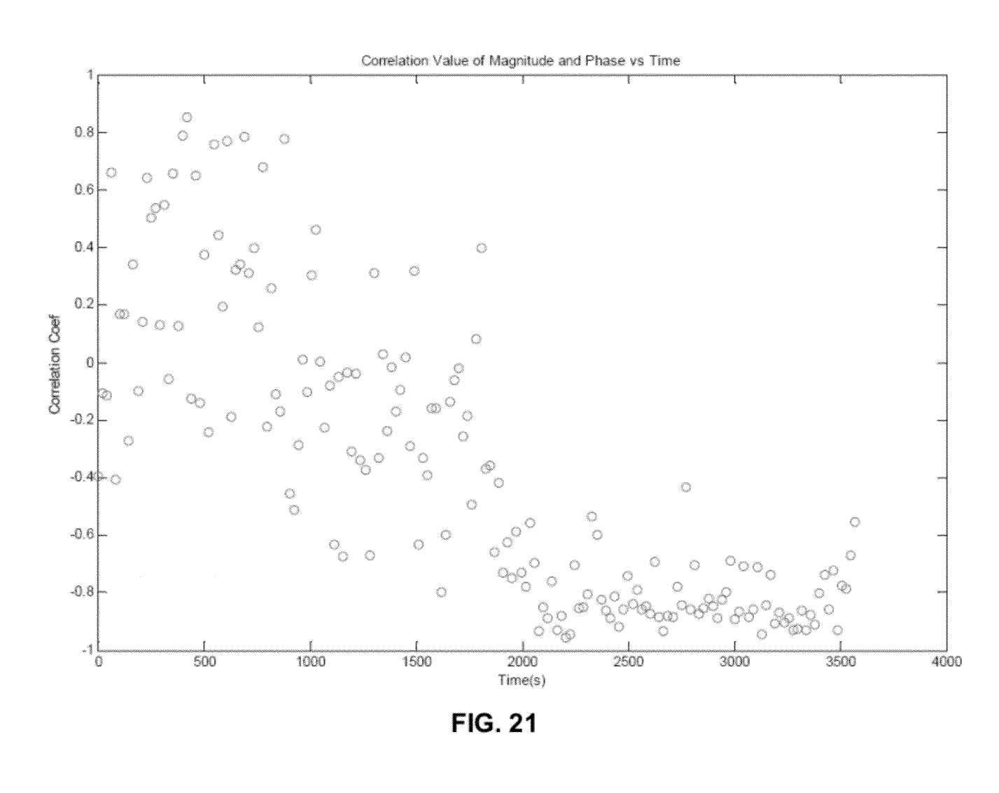

This phenomenon is displayed below in FIG. 21 in which over the course of one recording session, the correlation coefficient changes significantly. FIG. 21 is a plot showing how the correlation between phase and magnitude components changes after an eating event in the lesser curvature. The data was taken from a lesser curvature recording in canine 3. The eating event begins at 1000 seconds, and a clear downward trend can be observed heading toward a negative correlation coefficient value.

In some examples, the variability in impedance recordings may have come from the exact implant location, depth, and orientation. If the array of unipolar electrodes is experiencing sheer stress, or lateral (instead of axial) strain, the recording can be significantly impacted. The electrode implant locations were determined by visualizing approximate distances to physiological landmarks such as the pyloric sphincter. Inherently, this method is susceptible to error in the surgeon's judgment and changes in gastric anatomy from canine to canine.

As illustrated by the above, detecting the onset of feeding can be feasible using the phase of tissue impedance recorded from one or more locations of the stomach, such as, e.g., the fundus,. Based on the results, it appears that dynamic impedance can be measured using indwelling intramuscular electrodes placed the stomach. Further, changes in impedance occur during feeding may be reflected in by the real and imaginary components of impedance. The results may indicate that the onset of feeding correlates well with changes in impedance phase from electrodes in the fundus. The results may also indicate that the onset of feeding may also be detected in the antrum, but may require a longer time window to make an accurate determination.

Various aspects of the disclosure have been described. These and other aspects are within the scope of the following claims.

1. A method comprising:
determining a phase of tissue impedance at one or more gastrointestinal tract locations of a patient via a medical device; and
determining the occurrence of food intake by the patient based on the determined phase of the tissue impedance.

2. The method of claim 1, further comprising controlling electrical stimulation delivered to a gastrointestinal tract of the patient via the medical device based on the determined phase of the tissue impedance.

3. The method of claim 2, wherein controlling the electrical stimulation comprises initiating the delivery of electrical stimulation to the gastrointestinal tract of the patient.

4. The method of claim 2, wherein controlling the electrical stimulation comprises modifying at least one of an amplitude, frequency, or pulse width of the electrical stimulation to the gastrointestinal tract of the patient.

5. The method of claim 2, wherein controlling electrical stimulation delivered to the gastrointestinal tract of the patient comprises controlling electrical stimulation delivered to a stomach of the patient.

6. The method of claim 2, wherein the electrical stimulation comprises electrical stimulation configured to induce a feeling of at least one of satiety or nausea in the patient.

7. The method of claim 1, wherein the one or more gastrointestinal tract locations of the patient comprise one or more locations along a stomach of the patient.

8. The method of claim 1, wherein determining the occurrence of food intake by the patient based on the determined phase of the tissue impedance comprises comparing the phase of the tissue impedance to a threshold phase value and determining the occurrence of food intake by the patient based on the comparison.

9. The method of claim 1, wherein the phase on the tissue impedance comprises a phase angle of the tissue impedance.

10. The method of claim 1, further comprising storing food intake information in a memory based on the determined food intake by the patient.

11. The method of claim 1, wherein determining the phase of tissue impedance comprises:
sensing a signal generated in response to an applied signal; and
determining the phase of the tissue impedance based on the applied signal and the sensed signal.

12. The method of claim 11, wherein determining the phase of the tissue impedance based on the applied signal and the sensed signal comprises determining the phase of the tissue impedance based on a time delay between the applied signal and the sensed signal.

13. The method claim 1, wherein determining the occurrence of food intake by the patient based on the determined phase of the tissue impedance comprises determining the occurrence of food intake by the patient only based on the determined phase of the tissue impedance.

14. The method of claim 1, wherein determining the occurrence of food intake by the patient based on the determined phase of the tissue impedance comprises comparing frequency of a phase signal of the tissue impedance to a threshold frequency of a phase value and determining the occurrence of food intake by the patient based on the comparison.

15. The method claim 1, further comprising determining a magnitude of the tissue impedance, wherein determining the occurrence of food intake by the patient based on the determined phase of the tissue impedance comprises determining the occurrence of food intake by the patient based on the determined phase of the tissue impedance combined with the determined magnitude of the tissue impedance.

16. A medical device system comprising:
a sensing module configured to sense a signal at one or more gastrointestinal tract locations of a patient; and
a processor configured to determine a phase of tissue impedance at the one or more gastrointestinal tract locations, and determine the occurrence of food intake by the patient based on the determined phase of the tissue impedance.

17. The medical device system of claim 16, wherein the processor is configured to control delivery of electrical stimulation to a gastrointestinal tract the patient via the medical device based on the determined phase of the tissue impedance.

18. The medical device system of claim 17, wherein the processor controls the electrical stimulation by at least modifying at least one of an amplitude, frequency, or pulse width of the electrical stimulation to the gastrointestinal tract of the patient.

19. The medical device system of claim 17, wherein the processor controls the electrical stimulation by at least initiating the delivery of electrical stimulation to the gastrointestinal tract of the patient.

20. The medical device system of claim 17, wherein the processor controls electrical stimulation delivered to the gastrointestinal tract the patient by at least controlling electrical stimulation delivered to a stomach of the patient.

21. The medical device system of claim 17, wherein the electrical stimulation comprises electrical stimulation configured to induce a feeling of at least one of satiety or nausea in the patient.

22. The medical device system of claim 16, wherein the one or more gastrointestinal tract locations of the patient comprise one or more locations along a stomach of the patient.

23. The medical device system of claim 16, wherein the processor determines the occurrence of food intake by the patient based on the determined phase of the tissue impedance by at least comparing the phase of the tissue impedance to a threshold phase value and determining the occurrence of food intake by the patient based on the comparison.

24. The medical device system of claim 16, wherein the phase of the tissue impedance comprises a phase angle of the tissue impedance.

25. The medical device system of claim 16, further comprising a memory, wherein the processor is configured to store food intake information in the memory based on the determined food intake by the patient.

26. The medical device system of claim 16, wherein the sensing module senses the signal at one or more gastrointestinal tract locations by at least sensing a signal generated in response to an applied signal, wherein the processor determines the phase of tissue impedance by at least determining the phase of the tissue impedance based on the applied signal and the sensed signal.

27. The medical device system of claim 26, wherein the processor determines the phase of the tissue impedance based on the applied signal and the sensed signal by at least determining the phase of the tissue impedance based on a time delay between the applied signal and the sensed signal.

28. The medical device system of claim 16, wherein the processor is configured to determine the occurrence of food intake by the patient based on the determined phase of the tissue impedance by determining the occurrence of food intake by the patient only based on the determined phase of the tissue impedance.

29. The medical device system of claim 16, wherein the processor determines the occurrence of food intake by the patient based on the determined phase of the tissue impedance by at least comparing frequency of a phase signal of the tissue impedance to a threshold frequency of a phase value and determining the occurrence of food intake by the patient based on the comparison.

30. The medical device system of claim 16, wherein the processor is configured to determine a magnitude of the tissue impedance, wherein the processor determines the occurrence of food intake by the patient based on the determined phase of the tissue impedance by at least determining the occurrence of food intake by the patient based on the determined phase of the tissue impedance combined with the determined magnitude of the tissue impedance.

31. A system comprising:
means for determining a phase of tissue impedance at one or more gastrointestinal tract locations of a patient via a medical device; and
means for determining the occurrence of food intake by the patient based on the determined phase of the tissue impedance.

32. A non-transitory computer-readable storage medium comprising instructions to cause one or more programmable processors to:
determine a phase of tissue impedance at one or more gastrointestinal tract locations of a patient; and
determine the occurrence of food intake by the patient based on the determined phase of the tissue impedance.
